(19) United States
(12) Patent Application Publication
(10) Pub. No.: US 2024/0360518 A1
(43) Pub. Date: Oct. 31, 2024

(54) RADIOGENOMIC SIGNATURES IN BLADDER CANCER PATIENTS AND USES THEREOF

(71) Applicant: CEDARS-SINAI MEDICAL CENTER, Los Angeles, CA (US)

(72) Inventors: Charles J. Rosser, Valencia, CA (US); Hideki Furuya, Beverly Hills, CA (US); Simon Knott, Topanga, CA (US); Debiao Li, South Pasadena, CA (US); Xingyu Chen, Los Angeles, CA (US); Touseef Qureshi, Los Angeles, CA (US); Yibin Xie, West Hollywood, CA (US); Takashi Kobayashi, Kyoto (JP); Makito Miyake, Nara (JP)

(73) Assignee: CEDARS-SINAI MEDICAL CENTER, Los Angeles, CA (US)

(21) Appl. No.: 18/647,340

(22) Filed: Apr. 26, 2024

Related U.S. Application Data
(60) Provisional application No. 63/462,409, filed on Apr. 27, 2023.

(51) Int. Cl. C12Q 1/6886; A61K 45/06; C12Q 1/6869; G16H 30/40; G16H 50/20

(52) U.S. Cl. CPC C12Q 1/6886; A61K 45/06; C12Q 1/6869; G16H 30/40; G16H 50/20; C12Q 2600/112; C12Q 2600/158

(57) ABSTRACT

Provided herein are methods based on MRI data with genetic alterations of tumors in the determination of severity and provision of treatment options for bladder cancer patients.

### CROSS-REFERENCE TO RELATED APPLICATIONS

This application includes a claim of priority under 35 U.S.C. § 119 (e) to U.S. provisional patent application No. 63/462,409, filed Apr. 27, 2023, the entirety of which is hereby incorporated by reference.

### FIELD OF INVENTION

This invention relates to the use of both radiomics and genomic information to stage bladder cancer.

### BACKGROUND

Bladder cancer (BCa) is a common malignancy in the US. Muscle invasive BCa comprises approximately ⅓ of BCa and is associated with significant morbidity and mortality. Accurate staging of BCa (i.e., determining the extent of cancer) is essential to identifying the optimal treatment (e.g., choosing between radical cystectomy and bladder preservation). In the US, radical cystectomy is the treatment of choice of muscle invasive bladder cancer (MIBC) because of lingering data on bladder-preservation (BP) therapy being associated with decreased overall survival (OS). However, a patient's quality of life could be negatively affected by radical cystectomy, thus the continued intrigue with BP. Multiple BP options exist, although the approach of maximal transurethral resection of bladder tumor (TURBT) performed along with chemoradiation therapy (CRT) is the most favored. BP strategies need to develop and evolve if they are to address issues related to OS. Thus, a more thoughtful, phased approach to BP strategies need to develop and evolve in order to improve long-term outcomes for the growing need of patients who are ineligible for cisplatin based neoadjuvant chemotherapy and/or are either refusing or ineligible for radical cystectomy.

Existing National Comprehensive Cancer Network (NCCN) guidelines recommend CT scan of the abdomen and pelvis to assess the local extent of the cancer including involvement of regional lymph nodes. However, ⅓ of patients are understaged, while ⅓ are overstaged. Thus, what is desperately needed is a more accurate non-invasive modality to determine the extent of BCa.

It is an object of the present invention to provide methods of assaying, determining severity, or staging bladder cancers in a subject in need thereof and further treating the subject.

All publications herein are incorporated by reference to the same extent as if each individual publication or patent application was specifically and individually indicated to be incorporated by reference. The following description includes information that may be useful in understanding the present invention. It is not an admission that any of the information provided herein is prior art or relevant to the presently claimed invention, or that any publication specifically or implicitly referenced is prior art.

### SUMMARY OF THE INVENTION

The following embodiments and aspects thereof are described and illustrated in conjunction with compositions and methods which are meant to be exemplary and illustrative, not limiting in scope.

Methods for determining severity or stage of bladder cancer in a subject in need thereof and treating the subject in need thereof are provided, which include measuring in a biological sample of the subject an expression level of one or more marker genes of HOXB5, DHRS3, FABP4, TAGLN2, HIST1H1D, HIST1H2BD, H2AFX, CLDN3, and PLAUR; and/or performing radiomic analysis on a magnetic resonance (MR) image associated with the bladder of the subject by extracting from a region of interest (ROI) in the MR image one or more radiomic features of Energy, Cluster Prominence, Cluster Shade, Cluster Tendency, Homogeneity, Autocorrelation, Inverse Gaussian Left, Inverse Gaussian Left Focus, Gaussian Right Polar, Gaussian, Gaussian Right Focus, and Gaussian Right Polar; and providing a treatment comprising cystectomy to the subject if the subject is indicated as having a late stage bladder cancer, or providing a treatment comprising one or more of chemotherapy, radiotherapy, immunotherapy, and trans urethral resection of bladder tumour and excluding cystectomy if the subject is indicated as having an early stage bladder cancer. In some embodiments, the subject is indicated as having a late stage bladder cancer when the expression level of the one or more of the TAGLN2, the HIST1H1D, the HIST1H2BD, the H2AFX, the CLDN3, and the PLAUR, if measured, is higher, the expression level of the one or more of the HOXB5, the DHRS3, and the FABP4, if measured, is lower, and the extracted one or more radiomic features are higher, compared to respective ones in a reference subject having only a T0, T1, or T2 stage bladder cancer. In some embodiments, the subject is indicated as having an early stage bladder cancer when the expression level of the one or more of the HOXB5, the DHRS3, and the FABP4, if measured, is higher, the expression level of the one or more of the TAGLN2, the HIST1H1D, the HIST1H2BD, the H2AFX, the CLDN3, and the PLAUR, if measured, is lower, and the extracted one or more radiomic features are lower, compared to respective ones in a reference subject having a T3 or T4 stage bladder cancer.

In some embodiments, methods for determining severity or stage of bladder cancer in a subject in need thereof further include measuring in the biological sample expression levels of all or one or more genes from one or more of a plurality of gene sets selected from the group consisting of Basal, Basal Differentiation, Base Excision Repair, Cell Cycle, Homologous Recombination, MicroRNAs in Cancer, Oocyte Meiosis, p53 Signaling Pathway, Progesterone-mediated Oocyte Maturation, Pyrimidine Metabolism, Luminal, Luminal differentiation, Neuroendocrine differentiation, Normal Basal Intermediate, and Normal CDH12. In further embodiments, the expression levels of the all or one or more genes from each of the Basal, the Basal Differentiation, the Base Excision Repair, the Cell Cycle, the Homologous Recombination, the MicroRNAs in Cancer, the Oocyte Meiosis, the p53 Signaling Pathway, the Progesterone-mediated Oocyte Maturation, and the Pyrimidine Metabolism, gene sets] are higher in the subject indicated as having a late stage bladder cancer, compared to respective ones in the reference subject having only a T0, T1, or T2 stage bladder cancer. In further embodiments, the expression levels of the all or one or more genes from each of the Luminal, the Luminal differentiation, the Neuroendocrine differentiation, the Normal Basal Intermediate, and the Normal CDH12 are higher in the subject indicated as having an early stage bladder cancer, compared to respective ones in the reference subject having a T3 or T4 stage bladder cancer.

In some embodiments, the subject in need thereof is a human with suspected bladder tumor or human with a sessile appearing bladder mass.

In some embodiments, the biological sample comprises a bladder biopsy, a resected bladder tumor specimen, or a mucosal sample from cystectomy.

In some embodiments, the measuring of a gene expression level comprises performing single-cell RNA sequencing.

In some embodiments, a method for determining severity or stage of bladder cancer in a subject in need thereof and treating the subject includes measuring of the expression level in the biological sample and the providing of the treatment based on results from the measured expression level. In some embodiments, a method for determining severity or stage of bladder cancer in a subject in need thereof and treating the subject includes performing of the radiomic analysis and the providing of the treatment based on results from the radiomic analysis. In some embodiments, a method for determining severity or stage of bladder cancer in a subject in need thereof and treating the subject includes measuring of the expression level in the biological sample, performing of the radiomic analysis, and the providing of the treatment based on results from the measured expression level and radiomic analysis.

Methods are also provided for treating a subject in need thereof, including administering chemotherapy, radiation, immunotherapy, and/or performing transurethral resection of bladder tumor (TURBT), but not cystectomy, in a subject indicated as having an early stage bladder cancer, or performing cystectomy in a subject indicated as having a late stage bladder cancer, wherein the subject is indicated as having an early stage bladder cancer when expression level of one or more of genes HOXB5, DHRS3, and FABP4 in a biological sample of the subject is higher, expression level of one or more of genes TAGLN2, HIST1H1D, HIST1H2BD, H2AFX, CLDN3, and PLAUR in the biological sample is lower, and one or more radiomic features in a magnetic resonance image associated with a bladder of the subject is lower, compared to respective ones in a reference subject having a T3 or T4 stage bladder cancer, wherein the subject is indicated as having a late stage bladder cancer when expression level of one or more of the genes TAGLN2, HIST1H1D, HIST1H2BD, H2AFX, CLDN3, and PLAUR in the biological sample is higher, expression level of one or more of the genes HOXB5, DHRS3, and FABP4 in the biological sample is lower, and the one or more radiomic features in the magnetic resonance image associated with the bladder of the subject is higher, compared to respective ones in a reference subject having a T0, T1 or T2 stage bladder cancer, and wherein the one or more radiomic features comprises Energy, Cluster Prominence, Cluster Shade, Cluster Tendency, Homogeneity, Autocorrelation, Inverse Gaussian Left, Inverse Gaussian Left Focus, Gaussian Right Polar, Gaussian, Gaussian Right Focus, and Gaussian Right Polar.

In some embodiments, the subject indicated as having an early stage bladder cancer further has a higher expression level of all or one or more genes from one or more of gene sets Luminal, Luminal differentiation, Neuroendocrine differentiation, Normal Basal Intermediate, and Normal CDH12, and/or a low expression level of all or one or more genes from one or more of gene sets Basal, Basal Differentiation, Base Excision Repair, Cell Cycle, Homologous Recombination, MicroRNAs in Cancer, Oocyte Meiosis, p53 Signaling Pathway, Progesterone-mediated Oocyte Maturation, and Pyrimidine Metabolism, in the biological sample compared to respective ones in the reference subject having a T3 or T4 stage bladder cancer.

In some embodiments, wherein the subject indicated as having a late stage bladder cancer further has a higher expression level of all or one or more genes from one or more of gene sets Basal, Basal Differentiation, Base Excision Repair, Cell Cycle, Homologous Recombination, MicroRNAs in Cancer, Oocyte Meiosis, p53 Signaling Pathway, Progesterone-mediated Oocyte Maturation, and Pyrimidine Metabolism, and/or a low expression level of all or one or more genes from one or more of gene sets Luminal, Luminal differentiation, Neuroendocrine differentiation, Normal Basal Intermediate, and Normal CDH12, in the biological sample compared to respective ones in the reference subject having a T0, T1 or T2 stage bladder cancer.

Methods are further provided for monitoring response to a therapy and providing subsequent treatment in a subject with a bladder cancer, which include: providing the therapy to the subject with a bladder cancer; and i) measuring in a biological sample of the subject an expression level of one or more marker genes of HOXB5, DHRS3, FABP4, TAGLN2, HIST1H1D, HIST1H2BD, H2AFX, CLDN3, and PLAUR in response to the therapy; ii) performing radiomic analysis on a magnetic resonance (MR) image associated with the bladder of the subject in response to the therapy by extracting from a region of interest (ROI) in the MR image one or more radiomic features of Energy, Cluster Prominence, Cluster Shade, Cluster Tendency, Homogeneity, Autocorrelation, Inverse Gaussian Left, Inverse Gaussian Left Focus, Gaussian Right Polar, Gaussian, Gaussian Right Focus, and Gaussian Right Polar; and/or iii) measuring in the biological sample expression levels of all or one or more genes from one or more of a plurality of gene sets selected from the group consisting of Basal, Basal Differentiation, Base Excision Repair, Cell Cycle, Homologous Recombination, MicroRNAs in Cancer, Oocyte Meiosis, p53 Signaling Pathway, Progesterone-mediated Oocyte Maturation, Pyrimidine Metabolism, Luminal, Luminal differentiation, Neuroendocrine differentiation, Normal Basal Intermediate, and Normal CDH12 in response to the therapy; and
continuing the therapy, optionally in a lower dose than before, as a subsequent treatment for the subject if the expression level of the HOXB5, the DHRS3, and the FABP4 if measured has increased, the expression level of the TAGLN2, the HIST1H1D, the HIST1H2BD, the H2AFX, the CLDN3, and the PLAUR if measured has decreased, the one or more radiomic features if measured has lowered, the expression levels of the all or one or more genes from the gene sets Luminal, Luminal differentiation, Neuroendocrine differentiation, Normal Basal Intermediate, and Normal CDH12 if measured has increased, and the expression levels of the all or one or more genes from the gene sets Basal, Basal Differentiation, Base Excision Repair, Cell Cycle, Homologous Recombination, MicroRNAs in Cancer, Oocyte Meiosis, p53 Signaling Pathway, Progesterone-mediated Oocyte Maturation, and Pyrimidine Metabolism if measured has decreased, in response to the therapy, or
providing additional therapies, increasing doses, and/or replacing the therapy with radical cystectomy in the subject as subsequent treatment if the expression level of the TAGLN2, the HIST1H1D, the HIST1H2BD, the H2AFX, the CLDN3, and the PLAUR if measured has increased, the expression level of the HOXB5, the DHRS3, and the FABP4 if measured has decreased, the one or more radiomic features if measured has increased, the expression levels of the all or one or more genes from the gene sets Basal, Basal Differentiation, Base Excision Repair, Cell Cycle, Homologous Recombination, MicroRNAs in Cancer, Oocyte Meiosis, p53 Signaling Pathway, Progesterone-mediated Oocyte Maturation, and Pyrimidine Metabolism if measured has increased, and the expression levels of the all or one or more genes from the gene sets Luminal, Luminal differentiation, Neuroendocrine differentiation, Normal Basal Intermediate, and Normal CDH12 if measured has decreased, in response to the therapy.

Other features and advantages of the invention will become apparent from the following detailed description, taken in conjunction with the accompanying drawings, which illustrate, by way of example, various features of embodiments of the invention.

### BRIEF DESCRIPTION OF THE FIGURES

The patent or application file contains at least one drawing executed in color. Copies of this patent or patent application publication with color drawing(s) will be provided by the Office upon request and payment of the necessary fee.

Exemplary embodiments are illustrated in referenced figures. It is intended that the embodiments and figures disclosed herein are to be considered illustrative rather than restrictive.

FIGS. 1A and 1B depict intersection analysis of differential genes across three datasets. The upset plot illustrates the intersecting differential genes from train, test, and public databases. FIG. 1A represents the intersecting downregulated genes when comparing high- and low-stage samples. FIG. 1B demonstrates the intersecting upregulated genes when comparing high- and low-stage samples. The red color and dots symbolize the intersecting differential genes derived from the train, test, and public databases.

FIG. 1C depicts that there is no difference between BCa subtypes in HOXB5, DHRS3, and FABP4, which are highly expressed in early stage bladder cancer patients. FIG. 1D depicts the comparison between BCa subtypes in TAGLN2, PLAUR, and CLDN3, which are highly expressed in late stage bladder cancer patients. FIG. 1E depicts the comparison between BCa subtypes in H2AFX, HIST1H2BD, and HIST1H1D, which are highly expressed in late stage bladder cancer patients. The analysis revealed nine common genes, three out of which exhibited higher expression levels in low stage cases (lower expression levels in high stage): HOXB5, DHRS3, and FABP4, whereas six had higher expression levels in high stage cases (lower expression levels in low stage): TAGLN2, HIST1H1D, HIST1H2BD, H2AFX, CLDN3, and PLAUR. Only PLAUR demonstrated a significant difference between luminal and basal subtypes.

FIGS. 2A and 2B depict Luminal, Luminal_differentiation, Neuroendocrine_differentiation, Normal Basal_Intermediate, and Normal_CDH12 signatures, Basal, Basal_differentiation, Base_excision_repair, Cell_cycle, Homologous_recombination, MicroRNAs_in_cancer, Oocyte_meiosis, p53_signaling_pathway, Progesterone-mediated_oocyte_maturation, Proteasome, and Pyrimidine_metabolism.

FIGS. 2C-2E depict list of significant signatures: Basal, Basal_differentiation, Base_excision_repair, Cell_cycle, Homologous_recombination (2C); Luminal, Luminal_differentiation, MicroRNAs_in_cancer, Neuroendocrine_diferentiation, Normal_Basal_Intermediate, Normal_CDH12, Oocyte_meiosis, and p53_signaling_pathway (2D); and Progesterone-mediated_oocyte_maturation, Pyrimidine_metabolism (2E). 15 significant signatures associated with different stages of bladder cancer are highlighted in squares. *: p<0.05, **: p<0.01, ***: p<0.001, ****: p<0.0001 and ‘ns’: not significant.

FIG. 3 exemplifies radiomic analysis.

FIG. 4. Schematic diagram summarizing the workflow of radiogenomics assay.

### DESCRIPTION OF THE INVENTION

All references cited herein are incorporated by reference in their entirety as though fully set forth. Unless defined otherwise, technical and scientific terms used herein have the same meaning as commonly understood by one of ordinary skill in the art to which this invention belongs. Sambrook and Russel, Molecular Cloning: A Laboratory Manual 4^{th} ed., Cold Spring Harbor Laboratory Press (Cold Spring Harbor, NY 2012), provide one skilled in the art with a general guide to many of the terms used in the present application.

One skilled in the art will recognize many methods and materials similar or equivalent to those described herein, which could be used in the practice of the present invention. Indeed, the present invention is in no way limited to the methods and materials described. For purposes of the present invention, the following terms are defined below.

A “cancer” or “tumor” as used herein refers to an uncontrolled growth of cells which interferes with the normal functioning of the bodily organs and systems, and/or all neoplastic cell growth and proliferation, whether malignant or benign, and all pre-cancerous and cancerous cells and tissues. Included in this definition are benign and malignant cancers, as well as dormant tumors or micro-metastases. The term “invasive” refers to the ability to infiltrate and destroy surrounding tissue.

A subject can be one who has been previously diagnosed with or identified as suffering from or having a disease-state in need of monitoring (e.g., bladder cancer or bladder disease) or one or more complications related to such a disease-state, and optionally, have already undergone treatment for the disease-state or the one or more complications related to the disease/condition. Alternatively, a subject can also be one who has not been previously diagnosed as having a disease-state or one or more complications related to the disease/condition. For example, a subject can be one who exhibits one or more risk factors or symptoms for a disease-state (such as bladder cancer) or one or more complications related to a disease-state or a subject who does not exhibit risk factors. A “subject in need” can be a subject having that disease/condition, diagnosed as having that condition, or at risk of developing that disease. In some embodiments, a subject in need thereof is one having one or more bladder cancer symptoms or signs of bladder cancer. In some embodiments, a subject in need thereof is one having a sessile appearing bladder mass. In some embodiments, a subject in need thereof is one having bladder cancer and desires accurate staging and/or treatment of the bladder cancer. The terms, “patient”, “individual” and “subject” are used interchangeably herein. A “subject” means a human or animal. Usually the animal is a vertebrate such as a primate, rodent, domestic animal or game animal. In various embodiment, the subject is a human in the methods. In some embodiments, the subject is a human having bladder cancer. In further embodiments, the subject is a human having bladder cancer who does not have a urothelial cancer.

A non-cancer subject may be healthy and have no other disease, or they may have a disease other than cancer.

A biological marker (“biomarker” or “marker”) is a characteristic that is objectively measured and evaluated as an indicator of biologic processes, pathogenic processes, or pharmacological responses to therapeutic interventions. Typically markers are genes' or proteins' expression levels. Markers can also include patterns or ensembles of characteristics indicative of particular biological processes. In a broad sense, markers can also include radiomic features from medical imaging of a tissue. The biomarker measurement can increase or decrease to indicate a particular biological event or process. In addition, if the biomarker measurement typically changes in the absence of a particular biological process, a constant measurement can indicate occurrence of that process. A plurality of biomarkers includes at least two or more biomarkers (e.g., at least 2, 3, 4, 5, 6, and so on, in whole integer increments, up to all of the possible biomarkers) identified by the present invention, and includes any combination of such biomarkers.

The term “expression levels” refers to a quantity reflected in or derivable from the gene or protein expression data, whether the data is directed to gene transcript accumulation or protein accumulation or protein synthesis rates, etc. In some embodiments, the term “expression level” refers to the amount of gene transcript accumulation; and in some embodiments, the term “expression level” refers to the amount of protein accumulation; and in other embodiments, the term “expression level” refers to the amount of either gene transcript accumulation or protein transcript accumulation.

Gene markers may be polynucleotides that are genomic DNA, cDNA, or mRNA transcripts. The polynucleotide may contain deoxyribonucleotides, ribonucleotides, and/or their analogs and may be double-stranded or single stranded. A polynucleotide can comprise modified nucleic acids (e.g., methylated), nucleic acid analogs or non-naturally occurring nucleic acids and can be interrupted by non-nucleic acid residues. For example, a polynucleotide includes a gene, a gene fragment, cDNA, isolated DNA, RNA, tRNA, rRNA, isolated RNA of any sequence, recombinant polynucleotides, primers, probes, plasmids, and vectors. Additionally, the invention provides polynucleotides as markers that have substantial sequence similarity to a polynucleotide that is described by gene names herein. Two polynucleotides have “substantial sequence identity” when there is at least 80% sequence identity, at least 90% sequence identity, at least 95% sequence identity or at least 99% sequence identity between their amino acid sequences or when the polynucleotides are capable of forming a stable duplex with each other under stringent hybridization conditions. In some embodiments, the invention provides polynucleotides that have at least 95% sequence identity to HOXB5, DHRS3, FABP4, TAGLN2, HIST1H1D, HIST1H2BD), H2AFX, CLDN3, and PLAUR, or at least 95% sequence identity to those genes in the Basal gene set, the Basal Differentiation gene set, the Base Excision Repair gene set, the Cell Cycle gene set, the Homologous Recombination gene set, the MicroRNAs in Cancer gene set, the Oocyte Meiosis gene set, the p53 Signaling Pathway gene set, the Progesterone-mediated Oocyte Maturation gene set, the Pyrimidine Metabolism gene set, the Luminal gene set, the Luminal differentiation gene set, the Neuroendocrine differentiation gene set, the Normal Basal Intermediate gene set, and the Normal CDH12 gene set.

Polypeptides encoded by gene markers may reflect a single polypeptide appearing in a database. In general, the polypeptide is the largest polypeptide found in the database. But such a selection is not meant to limit the polypeptide to those corresponding to those single polypeptides. Accordingly, in another embodiment, the invention provides a polypeptide that is a fragment, or a homolog or allele of a marker. Additionally, the present invention includes polypeptides that have substantially similar sequence identity to the polypeptides encoded by the gene markers disclosed herein, or when polynucleotides encoding the polypeptides are capable of forming a stable duplex with each other under stringent hybridization conditions. For example, conservative amino acid substitutions may be made in polypeptides to provide functionally equivalent variants of the foregoing polypeptides, i.e., the variants retain the functional capabilities of the polypeptides. “Substantially sequence identity” refers to at least 80% sequence identity, at least 90% sequence identity, at least 95% sequence identity, at least 96% sequence identity, at least 97% sequence identity, at least 98% sequence identity, or at least 99% sequence identity to those polypeptides encoded by the gene markers disclosed herein.

Reagents or molecules which specifically bind the markers are also included. The term “specifically binding,” refers to the interaction between binding pairs (e.g., an antibody and an antigen or aptamer and its target). In some embodiments, the interaction has an affinity constant of at most 10⁻⁶ moles/liter, at most 10⁻⁷ moles/liter, or at most 10⁻⁸ moles/liter. In other embodiments, the phrase “specifically binds” refers to the specific binding of one protein to another (e.g., an antibody, fragment thereof, or binding partner to an antigen), wherein the level of binding, as measured by any standard assay (e.g., an immunoassay), is statistically significantly higher than the background control for the assay. For example, when performing an immunoassay, controls typically include a reaction well/tube that contain antibody or antigen binding fragment alone (i.e., in the absence of antigen), wherein an amount of reactivity (e.g., non-specific binding to the well) by the antibody or antigen binding fragment thereof in the absence of the antigen is considered to be background. Binding can be measured using a variety of methods standard in the art including enzyme immunoassays (e.g., ELISA), immunoblot assays, etc.).

In various embodiments, the level of the markers is compared to a standard level or a reference level. Typically, the standard biomarker level or reference range is obtained by measuring the same marker or markers in a set of normal controls. Measurement of the standard biomarker level or reference range need not be made contemporaneously; it may be a historical measurement. Preferably the normal control is matched to the patient with respect to some attribute(s) (e.g., age).

“Radiomics” generally refers to a process of extracting and analyzing mineable, quantitative data from radiographic images, with a goal of linking image features describing size, shape, and texture to the underlying tumor pathophysiology. In various embodiments, the radiomic analysis disclosed herein is used for prediction, prognostication or response monitoring in patients with bladder cancer, and particularly muscle invasive bladder cancer (MIBC).

The TNM system is a classification system to stage different types of cancer based on certain standards. Staging is the process of finding out how much cancer is in a person's body and where it's located. It's how the doctor determines the stage of a person's cancer. In the TNM system, each cancer is assigned a letter or number to describe the tumor (T), node (N), and metastases (M). T stands for the original (primary) tumor; N tells whether the cancer has spread to the nearby lymph nodes; and M tells whether the cancer has spread to distant parts of the body. Numbers after the T (such as T1, T2, T3, and T4) might describe the tumor size and/or amount of spread into nearby structures. The higher the T number, the larger the tumor and/or the more it has grown into nearby tissues.

The bladder is a hollow organ in the pelvis with flexible, muscular walls, where the body stores urine before it leaves the body. The bladder wall has many layers, made up of different types of cells. The inside lining of the bladder is urothelium or transitional epithelium. Urine is carried from the kidneys to the bladder through tubes called ureters. When muscles in your bladder contract, they push urine out through a tube called the urethra.

A person with bladder cancer will have one or more tumors in his/her bladder. Muscle invasive bladder cancer (MIBC) is a cancer that spreads into the detrusor muscle of the bladder. The detrusor muscle is the thick muscle deep in the bladder wall. Transitional cell carcinoma (sometimes also called urothelial carcinoma) is cancer that forms in the cells of the urothelium, where most bladder cancers start Symptoms of bladder cancer include hematuria (blood in the urine; often without pain), frequent an urgent need to pass urine, pain when passing urine, pain in the lower abdomen, and back pain.

Conventionally, the stage of bladder cancer can be identified from biopsies that are often done with transurethral resection of bladder tumor (TURBT), a procedure for tumor typing, staging and grading. The stages of bladder cancer are generally: i) Ta: tumor on the bladder lining that does not enter the muscle, ii) Tis: carcinoma in situ, looking like a reddish, velvety patch on the bladder lining, iii) T1: tumor goes through the bladder lining (e.g., tumor found in the lamina propria) but does not reach the muscle layer, iv) T2: tumor grows into the muscle layer of the bladder, v) T3: tumor goes past the muscle layer into tissues around the bladder, and vi) T4: tumor has spread to nearby structures such as lymph nodes and the prostate in men or the vagina in females. Generally, Ta, Tis and T1 are non-muscle-invasive bladder cancer (NMIBC), while T2, T3 and T4 are muscle-invasive bladder cancer (MIBC). In some embodiments, an early stage (also referred to as a low-stage) bladder cancer as indicated with expression markers and/or radiomic features disclosed herein refers to T1 or T2 bladder cancer, or optionally Ta or Tis; whereas a late stage (also referred to as a high-stage) bladder cancer as indicated with expression markers and/or radiomic features disclosed herein refers to T3 or T4 bladder cancer.

In some embodiments, radiomic analysis is performed based on images obtained from MRI with T2-weighted (T2W) image, diffusion weighted images (DWI), and/or dynamic contrast-enhanced image (DCE MRI). Preferably the MRI possesses the ability to better delineate cancer within the bladder and its surrounding area (e.g., lymph nodes).

In some embodiments, radiomic analysis includes operations of one or more of:
accessing a multi-parametric magnetic resonance imaging (MRI) image associated with a patient, or operating a multi-parametric MRI to obtain MRI image associated with the patient, where the image includes a region of interest (ROI) demonstrating bladder cancer pathology, the image having a plurality of pixels, a pixel having an intensity;
segmenting a tumoral region represented in the image, where segmenting the tumoral region includes defining a tumoral boundary;
extracting a set of radiomic features from the image, where the set of radiomic features includes at least one radiomic feature extracted within the tumoral region and/or from a peritumoral region;
providing the set of radiomic features to a machine learning classifier trained to distinguish a first class from a second, different class based on the set of radiomic features;
receiving, from the machine learning classifier, a probability that the ROI is a member of the first class, where the machine learning classifier computes the probability based on the set of radiomic features;
classifying the ROI as a member of the first class or the second, different class based, at least in part, on the probability; and
displaying the classification.

In some embodiments, different image types, sequences or imaging series (e.g. CT, MRI, PET) can be used individually or in combination in the methods and processes of the invention.

In some embodiments, the machine learning classifier is a quadratic discriminant analysis (QDA) classifier.

In some embodiments, the first class is intra-vesical bladder cancer (e.g., T1 or T2 stage bladder cancer), and where the second class is extra-vesical bladder cancer (e.g., T3 or T4 stage bladder cancer).

Exemplary radiomic features and their extraction from MRI images (e.g., T2W images) are described in US20200005931 and U.S. Pat. No. 10,854,338, which are incorporated by reference herein. For example, the University College in London (UCL) has developed a software platform known as TexRAD that provides quantitative measurements (referred to herein as Quantitative Textural Analysis or QTA) of tumor lesions present on images. QTA is a post-processing technique that can be used to quantify tissue complexity by assessing the distribution of textural features (or heterogeneity) within a tumor lesion and their change following treatment.

In some embodiments, radiomic features include tumor heterogeneity. Tumor complexity can be quantified by QTA using a range of measurable parameters based on enhancement characteristics and/or density changes on a local level by clustering small groups of pixels together using filter kernels (referred to as spatial scale filters (SSF)) within a lesion itself. The output from the analysis then provides a measure of tumor heterogeneity. However, much of the heterogeneity visible on a radiological image can represent photon noise, which tends to mask or suppress the signal strength of underlying biologic information. By first filtering out the noise, QTA analysis can then be used to more effectively probe the biological diversity inherent in tumor complexity.

In some embodiments, a QTA parameter, entropy, measures the mean density of clustered pixels within the ROI, i.e. irregularity in the ROI; which serves as a measure of heterogeneity.

In some embodiments, standard deviation (SD) measures the spread of density distribution in the filtered image. The natural logarithm of mean pixel density normalized to the total number of pixels. It is a measure of heterogeneity and microstructural changes in the entire ROI.

In some embodiments, raw MRI images for example in the DICOM/NII format is input into an algorithm to extract features from a cancer region (e.g., region of interest, ROI, identified by a trained radiologist), and the features may be converted to quantities (or numbers). Then the quantities were evaluated by statistical analysis such as student's t test. In some embodiments, each of the radiomics features represent a combination of signal intensities of pixels/voxels in the ROI. For example in our study, we have identified from a statistical comparison of features from known higher stage bladder cancer and from known lower stage bladder cancer that each of Heterogeneity, Cluster Difference, Inverse Gaussian, and Quantile 25 is significantly higher in the higher-stage bladder cancer than in the lower-stage bladder cancer.

In various embodiments, intra-vesical (T0, T1, T2) bladder cancer and extra-vesical (T3, T4, or N+ or M+) bladder cancer have differential gene expressions. For example, intra-vesical BC has differential (increased) gene expression of HOXB5, DHRS3, and FABP4, compared to extra-vesical BC; and extra-vesical BC has differential (increased) gene expression of TAGLN2, HIST1H1D, HIST1H2BD, H2AFX, CLDN3, and PLAUR, compared to intra-vesical BC or compared to a normal urothelium.

HOXB5 gene encodes homeobox B5. DHRS3 gene encodes dehydrogenase/reductase 3. FABP4 gene encodes fatty acid binding protein 4. TAGLN2 gene encodes transgelin-2. HIST1H1D gene encodes histone H1.3. HIST1H2BD) gene encodes histone H2B type 1-D. H2AFX gene encodes H2A histone family member X. CLDN3 gene encodes claudin 3. PLAUR gene encodes plasminogen activator urokinase receptor. Further gene acronyms or symbols herein are consistent with conventions in the art, and detailed information can be accessed in databases such as the National Center for Biotechnology Information (NIH).

In some embodiments, a higher (or lower) gene expression level of a set of genes refers to the higher (or lower) expression level of each of the genes in the set. In some embodiments, a higher (or lower) gene expression level of a set of genes refers to a higher (or lower) score calculated from a mathematical formula including one or more, or preferably 50% or more, or all of the genes in the set. In some embodiments, a higher gene expression level of a gene disclosed herein is at least a LogFoldChange of 10 or greater than a reference or a control.

In further embodiments, extra-vesical BC or more severe dysplasia has increased gene expression of the following gene sets, compared to intra-vesical or less severe dysplasia or compared to normal urothelium:
Basal: CD44, CDH3, KRT1, KRT14, KRT16, KRT5, KRT6A, KRT6B, KRT6C, SFN, S100A8, PI3, S100A7, CSTA, SERPINB2, CKS2, CONB1, CC2, KIF23, and PMAIP1.
Basal Differentiation: LYNX1, SCEL, CTB-113P19.4, S100A8, SLP1, S100A9, PI3, CEACAM6, VSNL1, SERPINB2, CSTA, FGFBP1, SPRR1B, DSG3, KRT1, S100A7, CLCA2, LGALS7, SPRR2B, KRT6A, KRT6B, SFN, SERPINB3, KRT16, SERPINB3, SERPINB13, SERPINB4, KRT4, SPRR1A, KRT6A, ABCA12, SPRR3, and TME.
Base Excision Repair: FEN1, LIG1, NEIL3, PARP1, PARP2, PCNA (encoding for proliferating cell nuclear antigen), POLE, POLE2, and UNG.
Cell Cycle: BUB1, BUB1B, CC2, CONB2, CCNE1, CCNE2, CDC20, CDC25A, CDC25C, CDC6, CDK1, CDK2, CDKN2A, DBF4, E2F1, E2F2, ESPL1, MAD2L1, MAD2L2, MCM2, MCM4, MCM6, MCM7, ORC1, ORC6, PC, PLK1, SKP2, SMC3, TFDP1, TTK, YWHAB, ABL1, APC1, APC10, APC11, APC13, APC2, APC4, APC5, APC7, ATM, ATR, BUB1, BUB1B, BUB3, CC1, CC2, CONB1, CONB2, CCNB3, COND1, CCND2, CCND3, CCNE1, CCNE2, CONH, CDC14A, CDC14B, CDC16, CDC20, CDC23, CDC25A, CDC25B, CDC25C, CDC26, CDC27, and CD.
Homologous Recombination: BLM, BRCA2, EME1, RAD51, RAD51C, RAD54L, RPA1, RPA3, TOP3A, XRCC2, and XRCC3.
MicroRNAs in Cancer: BRCA1, CCNE1, CCNE2, CDC25A, CDC25C, CDCA5, CDKN2A, DNMT1, E2F1, E2F2, EZH2, KIF23, STMN1, and TRIM71.
Oocyte Meiosis: AURKA, BUB1, CONB2, CCNE1, CCNE2, CDC20, CDC25C, CDK1, CDK2, ESPL1, FBXO5, MAD2L1, MAD2L2, PLK1, SGOL1, SMC3, and YWHAB.
p53 Signaling Pathway: CCNB2, CCNE1, CCNE2, CDK1, CDK2, CDKN2A, GTSE1, PPMID, RFWD2, and RRM2.
Progesterone.mediated Oocyte Maturation: BUB1, CCNA2 (encoding for cyclin A2), CCNB2, CDC25A, CDC25C, CDK1, CDK2, MAD2L1, MAD2L2, and PLK1.
Pyrimidine Metabolism: (TPS1, DTYMK, POLA2, POLE, POLE2, PRIM1, PRIM2, RRM2, and TYMS.

In further embodiments, lower-stage BC or less severe dysplasia has increased gene expression of the following gene sets, compared to higher-stage or more severe dysplasia:
Luminal: CYP2J2, ERBB2, ERBB3, FGFR3, FOXA1, GATA3, GPX2, KRT18, KRT19, KRT20, KRT7, KRT8, PPARG, XBP1, PEPD, ACADM, MGST1, CYP4B1, ACAA1, CEBPA, ACOX1, ACSL1, SONNIG, ELOVL6, HES1, SDC1, IGFBP3, and GDF15.
Luminal Differentiation: FGFR3, GATA3, FOXA1, UPK2, PPARG, KRT20, SNX31, and UPK1A.
Neuroendocrine Differentiation: ZDHHC15, FAM184A, CAC1A, EML5, SP25, CAC2D2, DCX, PSIP1, ST18, RGS7, INSM1, SLC4A8, CAMK2B, ASCL1, RIN1, SCN3A, ADAM22, FAM105A, DPY19L2P2, ASRGL1, SLCIA2, MAP6, NRXN1, TTLL7, CNKSR2, RAB39B, KONC1, GKAP1, TMEM170B, PPM1E, ELAVL4, STXBP5L, and GPR137C.
Normal CDH12: LINC00486, RBFOX1, RASGEFIB, CSMD1, CNTP2, INO80D, SLC26A3, SLC27A6, EYS, CTN3, PTPRD, PCDH15, LRP1B, CTN2, GALNTL6, CALN1, ROBO2, GRID2, NRXN3, AC007402.1, GPC5, NRXN1, DPP10, CNTN5, AGBL4, LRRC4C, DLGAP1, FP236383.1, CDH13, TMEM132D, TENM2, SGCZ, OPCML, CDH18, ERC2, NRG3, DSCAM, CSMD3, AL589740.1, DLG2, CACIA, DCC, CNTP5, AC011287.1, CCDC26, CADM2, AGBL1, PTPRT, KCNIP4, AC109466.1, NRG1, HBB, CDH12, LINC02240, TRPM3, CTNND2, ADAMTSL1, FP700111.1, MDGA2, SNTG1, SORCS1, AC113414.1, LUZP2, GRM7, ZNF385D, LINGO1, NTM, ASIC2, FSTL5, HBA2, NELL1, LRRTM4, THSD7B, SLC30A10, SYN3, LINC01090, USH2A, LSAMP, DLGAP2, CSMD2, ADGRL3, PKN2-AS1, CNTN4, ERVMER61-1, MIR4300HG, CPNE4, XKR4, LINC00276, CNBD1, and HYDIN.
Normal Basal Intermediate: DGKH, LINC00511, PTPRQ, MALAT1, ABCC3, NEAT1, ITGA6, COL4A5, ELF3, MECOM, USP31, FSTL4, FRMD6, KRT13, MYOF, CCSER1, TMPRSS4, ACOXL, SCHLAP1, DST, MCC, KIAA1217, PTPRM, CYP3A5, PTPN13, C16orf74, CRYBG1, RASSF6, PTK2, ATP8B1, ITGA3, KRT19, NEDD4L, DHRS3, VWA8, SLC14A1, OXR1,9-Sep, CMIP, BTBD16, AC013652.1, SRGAP3, PPARG, AC044810.3, GRHL2, ITGB6, MSI2, MPPED2, ATF7IP2, IL1RAPL2, MAST4, BBOX1-AS1, AHR, ATF3, CLCA4, ID1, FAM160A1, DTNB, RIMS2, KANK1, SMAD3, MET, ADK, SPIDR, MYO1E, SAMD12, PARD3, SEMA4B, ITGB4, PPFIBP1, ETS2, HOTAIRM1, PATJ, LINC01876, TP63, MIR4713HG, TMC7, CYP24A1, NSUN6, CA12, KRT17, AC009478.1, BHLHE40, SLC26A5, MTCL1, AKRIC2, CROT, ITGA2, ADGRF1, NHS, MCTP2, CAPN8, SCN11A, ARHGAP32, CHST9, KLHL29, GCC2, ZBTB7C, PTPRK, COBLL1, EXOC6B, and RAB10.

### Diagnosis & Treatment of Extra-Vesicle (Late-Stage) Bladder Cancer

In some embodiments, a method is provided for assaying a urothelial/bladder tissue in a subject in need thereof, e.g., for staging (determining stage or severity of) bladder cancer in the subject in need thereof, and optionally providing treatment therefor, which include measuring in a urothelial/bladder tissue sample an expression level of one or more of HOXB5, DHRS3, FABP4, TAGLN2, HIST1H1D, HIST1H2BD, H2AFX, CLDN3, and PLAUR, wherein (i) a higher expression level of TAGLN2, HIST1H1D, HIST1H2BD, H2AFX, CLDN3, and PLAUR, each one if measured, (ii) a lower expression level of HOXB5, DHRS3, and FABP4, each one if measured, or both (i) and (ii), compared to respective levels in a subject having a low stage bladder cancer such as T0, T1, or T2, indicate that the subject in need thereof has an extra-vesicle bladder cancer (e.g., T3 or T4).

In some embodiments, an average being higher than a reference level of the (normalized) gene expression levels of all, or at least 95%, 90%, 85%, 80%, 75%, 70%, 60%, or 50%, of the genes in each one of the ten, or any one, two, three, four, five, six, sever, eight, or nine, of the Basal gene set, the Basal Differentiation gene set, the Base Excision Repair gene set, the Cell Cycle gene set, the Homologous Recombination gene set, the MicroRNAs in Cancer gene set, the Oocyte Meiosis gene set, the p53 Signaling Pathway gene set, the Progesterone-mediated Oocyte Maturation gene set, and the Pyrimidine Metabolism gene set indicates that the subject has a stage III (T3) or IV (T4) bladder cancer, wherein the reference level is an average of respective (normalized) gene expression levels detected from a sample of a subject with normal urothelium or of a subject with intra-vesical (T1 or T2) bladder cancer. In some instances for a plurality of genes, a normalized expression level is used for each gene in order to compute an average, e.g., an average is the sum of each gene's normalized expression level divided by the number of genes. For example, for determining or indicating a high-stage bladder cancer, the expression level for a gene in a known extra-vesical bladder cancer sample (e.g., T4 bladder cancer) is set as 100%, and the expression level for the gene in a tested sample is normalized against that in the known high-stage bladder cancer sample. Alternatively, for determining or indicating a high-stage bladder cancer, the expression level for a gene in a known normal urothelium is set as 100%, and the expression level for the gene in a tested sample is normalized against that in the normal urothelium sample.

In some embodiments, expression levels being higher than respective reference levels in all, or at least 95%, 90%, 85%, 80%, 75%, 70%, 60%, or 50%, of the genes in each one of the ten, or any one, two, three, four, five, six, sever, eight, or nine, of the Basal gene set, the Basal Differentiation gene set, the Base Excision Repair gene set, the Cell Cycle gene set, the Homologous Recombination gene set, the MicroRNAs in Cancer gene set, the Oocyte Meiosis gene set, the p53 Signaling Pathway gene set, the Progesterone-mediated Oocyte Maturation gene set, and the Pyrimidine Metabolism gene set indicates that the subject has a stage III (T3) or IV (T4) bladder cancer, wherein reference level is that detected from a sample of a subject with normal urothelium or of a subject with intra-vesical (T1 or T2) bladder cancer.

In some embodiments, a subject indicated to have a stage III (T3) or IV (T4) bladder cancer also has an average being lower than a reference level of the (normalized) gene expression levels of all, or at least 95%, 90%, 85%, 80%, 75%, 70%, 60%, or 50%, of the genes in each one of the five, or any one, two, three, or four, of the Luminal gene set, the Luminal differentiation gene set, the Neuroendocrine differentiation gene set, the Normal Basal Intermediate gene set, and the Normal CDH12 gene set, wherein the reference level is an average detected from a sample of a subject with normal urothelium or of a subject with intra-vesical (T1 or T2) bladder cancer. Hence, in some embodiments, an average being lower than a reference level of (normalized) gene expression levels of all, or at least 95%, 90%, 85%, 80%, 75%, 70%, 60%, or 50%, of the genes in each one of the five, or any one, two, three, or four, of the Luminal gene set, the Luminal differentiation gene set, the Neuroendocrine differentiation gene set, the Normal Basal Intermediate gene set, and the Normal CDH12 gene set indicates the subject has a stage III (T3) or IV (T4) bladder cancer, wherein the reference level is an average detected from a sample of a subject with normal urothelium or of a subject with intra-vesical (T1 or T2) bladder cancer.

In some embodiments, a subject indicated to have a stage III (T3) or IV (T4) bladder cancer also has expression levels being lower than respective reference levels in all, or at least 95%, 90%, 85%, 80%, 75%, 70%, 60%, or 50%, of the genes in each one of the five, or any one, two, three, or four, of the Luminal gene set, the Luminal differentiation gene set, the Neuroendocrine differentiation gene set, the Normal Basal Intermediate gene set, and the Normal CDH12 gene set, wherein reference level is that detected from a sample of a subject with normal urothelium or of a subject with intra-vesical (T1 or T2) bladder cancer. Hence, in some embodiments, expression levels being lower than respective reference levels in all, or at least 95%, 90%, 85%, 80%, 75%, 70%, 60%, or 50%, of the genes in each one of the five, or any one, two, three, or four, of the Luminal gene set, the Luminal differentiation gene set, the Neuroendocrine differentiation gene set, the Normal Basal Intermediate gene set, and the Normal CDH12 gene set, indicates that the subject has a stage III (T3) or IV (T4) bladder cancer, wherein reference level is that detected from a sample of a subject with normal urothelium or of a subject with intra-vesical (T1 or T2) bladder cancer.

Further embodiments provide that methods of assaying a urothelial/bladder tissue in a subject in need thereof, e.g., for staging (determining stage or severity of) bladder cancer in the subject in need thereof and optionally providing treatment therefor, include obtaining or performing a magnetic resonance (MR) scan to obtain an image of a urothelial tissue or bladder of the subject, and measuring in a region of interest (ROI) comprising or consisting of a tumor in the urothelial tissue or the bladder one or more radiomic features comprising Energy, Cluster Prominence, Cluster Shade, Cluster Tendency (also known as Cluster Trend), Homogeneity, Autocorrelation, Inverse Gaussian Left, Inverse Gaussian Left Focus, Gaussian Right Polar, Gaussian, Gaussian Right Focus, and Gaussian Right Polar.

Preferably, the MR scan is a T2-weighted scan. In other embodiments, the MR scan is T2 map, T2* map, T2 or T2* map, T2 or T2*-weighted, corrected T2 or T2*-weighted scan.

Alternatively the methods include performing or obtaining a CT scan of a urothelial tissue of bladder and measuring the one or more radiomic features in an ROI comprising or consisting of or outlining a tumor.

Radiomic features Energy, Cluster Prominence, Cluster Shade, Cluster Trend (also known as Cluster Tendency), Homogeneity, and Autocorrelation are described by the Imaging Biomarker Standardization Initiative (IBSI) in Zwanenburg et al., In eprint arXiv: 1612.07003, 2016, as well as in open-source database such as pyradiomics.readthedocs.io/en/latest/features.html#. In various instances, they are extracted (calculated) using PyRadiomics based on images such as MR images, either the original images and/or a derived image obtained by applying a filter.

Specifically, radiomic feature Energy is a measure of the magnitude of voxel values in an image. Typically a larger values implies a greater sum of the squares of these values. Energy is a first order feature, i.e., belonging to first-order statistics that describe the distribution of voxel intensities within the image region defined by the mask through basic metrics. For example, let: X be a set of Nₚ voxels included in the ROI, P(i) be the first order histogram with N_{g} discrete intensity levels, where N_{g} is the number of non-zero bins, equally spaced from 0 with a width defined in the bin Width parameter; and p(i) be the normalized first order histogram and equal to P(i)/Nₚ, and optionally voxelArrayShift [0] be Integer (added to the gray level intensity to prevent negative values) Energy is represented by formula (Ia):

$$energy = {\sum }_{i = 1}^{{N}_{p}} {( X ( i ) + c )}^{2}$$

wherein c is optional value (e.g., defined by voxel Array Shift in PyRadiomics open-source database), which shifts the intensities to prevent negative values in X, X is gray level intensity. This ensures that voxels with the lowest gray values contribute the least to Energy, instead of voxels with gray level intensity closest to 0.

Cluster Prominence, Cluster Shade, Cluster Tendency, Homogeneity, and Autocorrelation are gray level co-occurrence matrix (GLCM) features. A GLCM of size N_{g}×N_{g} describes the second-order joint probability function of an image region constrained by the mask and is defined as P(i,j|δ, θ). The (i, j)^{th} element of this matrix represents the number of times the combination of levels i and j occur in two pixels in the image, that are separated by a distance of δ pixels along angle θ. The distance δ from the center voxel is defined as the distance according to the infinity norm. For δ=1, this results in 2 neighbors for each of 13 angles in 3D (26-connectivity) and for δ=2 a 98-connectivity (49 unique angles). For example, let P(i, j) be the co-occurrence matrix for an arbitrary δ and θ; p(i, j) be the normalized co-occurrence matrix and equal to P(i, j)/ΣP(i,j); N_{g} be the number of discrete intensity levels in the image; pₓ(i)=Σⱼ₌₁^{N}^{_{g}}p(i, j) be the marginal row probabilities; p_{y}(i)=Σᵢ₌₁^{N}^{_{g}}p(i, j) be the marginal column probabilities; μₓ be the mean gray level intensity of pₓ and defined as μₓ=Σᵢ₌₁^{N}^{_{g}}pₓ(i)i; and μ_{y} be the mean gray level intensity of p_{y} and defined as μ_{y}=Σⱼ₌₁^{N}^{_{g}}p_{y}(j)j.

Radiomic feature Autocorrelation is a measure of the magnitude of the fineness and coarseness of texture. Autocorrelation is represented by formula (Ib):

$$autocorrelation = {\sum }_{i = 1}^{{N}_{g}} {\sum }_{j = 1}^{{N}_{g}} p ( i , j ) i j$$

Cluster Prominence is a measure of the skewness and asymmetry of the GLCM. A higher values implies more asymmetry about the mean while a lower value indicates a peak near the mean value and less variation about the mean. Cluster Prominence is represented by formula (Ic):

$$cluster prominence = {\sum }_{i = 1}^{{N}_{g}} {\sum }_{j = 1}^{{N}_{g}} {( i + j - {μ}_{x} - {μ}_{y} )}^{4} p ( i , j )$$

Cluster Shade is a measure of the skewness and uniformity of the GLCM. A higher clustershade implies greater asymmetry about the mean. Cluster Shade is represented by formula (Id):

$$cluster shade = {\sum }_{i = 1}^{{N}_{g}} {\sum }_{j = 1}^{{N}_{g}} {( i + j - {μ}_{x} - {μ}_{y} )}^{3} p ( i , j )$$

Cluster Tendency is a measure of groupings of voxels with similar gray-level values. Cluster Tendency is represented by formula (Ie):

$$cluster tendency = {\sum }_{i = 1}^{{N}_{g}} {\sum }_{j = 1}^{{N}_{g}} {( i + j - {μ}_{x} - {μ}_{y} )}^{2} p ( i , j )$$

Homogeneity is represented by formula (If):

$$homogeneity = {\sum }_{i = 1}^{{N}_{g}} {\sum }_{j = 1}^{{N}_{g}} \frac{p ( i , j )}{1 + \vert i - j \vert}$$

Radiomic feature Inverse Gaussian Left is represented by formula (IIa):

$$Inverse Gaussian Left = {\sum }_{i = 1}^{n} {\sum }_{j = 1}^{n} {temp}_{ij} \cdot {glcm}_{ij}$$

wherein i, j represents the corresponding row and column in the glcm matrix, n is the dimension of the glcm matrix, and tempᵢⱼ is

$$\frac{1}{exp ( - \frac{{( i - μ )}^{2} + {( j - μ )}^{2}}{2 {σ}^{2}} )} ,$$

wherein σ=std([1, 2, . . . , n]) is the standard deviation of the values from 1 to n, and μ=1; and if glcm is not dianoal, then tempᵢⱼ=0 from 1 to n.

Radiomic feature Inverse Gaussian Left Focus is represented by formula (IIb):

$$Inverse Gaussian Left Focus = {\sum }_{i = 1}^{n} {\sum }_{j = 1}^{n} {temp}_{ij} \cdot {glcm}_{ij}$$

wherein i, j represents the corresponding row and column in the glcm matrix, n is the dimension of the glcm matrix, and tempᵢⱼ is

$$\frac{1}{exp ( - \frac{{( i - μ )}^{2} + {( j - μ )}^{2}}{2 {σ}^{2}} )} ,$$

wherein σ=std([1, 2, . . . , n]) is the standard deviation of the values from 1 to n, and μ=median (1, . . . , median (1, . . . , n)) is the median of consecutive numbers from 1 to the median of consecutive numbers from 1 to n, with the first stage median round down; and if glcm is not dianoal, then tempᵢⱼ=0 from 1 to n.

Radiomic feature Inverse Gaussian Right Polar is represented by formula (IIc):

$$Inverse Gaussian Right Polar = {\sum }_{i = 1}^{n} {\sum }_{j = 1}^{n} {temp}_{ij} \cdot {glcm}_{ij}$$

wherein i, j represents the corresponding row and column in the glcm matrix, n is the dimension of the glcm matrix, and tempᵢⱼ is

$$\frac{1}{exp ( - \frac{{( i - μ )}^{2} + {( j - μ )}^{2}}{2 {σ}^{2}} )} ,$$

wherein σ=std([1, 2, . . . , n]) is the standard deviation of the values from 1 to n, and μ=n; and if glcm is not dianoal, then tempᵢⱼ=0 from 1 to n.

Radiomic feature Gaussian Right Polar is represented by formula (IId):

$$Gaussian Right Polar = {\sum }_{i = 1}^{n} {\sum }_{j = 1}^{n} {temp}_{ij} \cdot {glcm}_{ij}$$

wherein i, j represents the corresponding row and column in the glcm matrix, n is the dimension of the glcm matrix, and tempᵢⱼ is exp

$$( - \frac{{( i - μ )}^{2} + {( j - μ )}^{2}}{2 {σ}^{2}} ) ,$$

wherein σ=std([1, 2, . . . , n]) is the standard deviation of the values from 1 to n, and μ=n; and if glcm is not dianoal, then tempᵢⱼ=0 from 1 to n.

Radiomic feature Gaussian Right Focus is represented by formula (IIe):

$$Gaussian Right Focus = {\sum }_{i = 1}^{n} {\sum }_{j = 1}^{n} {temp}_{ij} \cdot {glcm}_{ij}$$

wherein i, j represents the corresponding row and column in the glcm matrix, n is the dimension of the glcm matrix, and tempᵢⱼ is exp

$$( - \frac{{( i - μ )}^{2} + {( j - μ )}^{2}}{2 {σ}^{2}} ) ,$$

wherein σ=std([1, 2, . . . , n]) is the standard deviation of the values from 1 to n, and μ=median (round (median (1, . . . , n), . . . , n) is the median of consecutive numbers between n and the median of consecutive numbers from 1 to n, which the first stage median is rounded to nearest integer; and if glcm is not dianoal, then tempᵢⱼ=0 from 1 to n.

Radiomic feature Gaussian is represented by formula (IIf):

$$Gaussian = {\sum }_{i = 1}^{n} {\sum }_{j = 1}^{n} {temp}_{ij} \cdot {glcm}_{ij}$$

wherein i, j represents the corresponding row and column in the glcm matrix, n is the dimension of the glcm matrix, and tempᵢⱼ is exp

$$( - \frac{{( i - μ )}^{2} + {( j - μ )}^{2}}{2 {σ}^{2}} ) ,$$

wherein σ=std([1, 2, . . . , n]) is the standard deviation of the values from 1 to n, and μ=mean (1, . . . , n) is the mean of consecutive numbers from 1 to n; and if glcm is not dianoal, then tempᵢⱼ=0 from 1 to n.

In various aspects, these radiomic features of Energy, Cluster Prominence, Cluster Shade, Cluster Tendency (also known as Cluster Trend), Homogeneity, Autocorrelation, Inverse Gaussian Left, Inverse Gaussian Left Focus, Gaussian Right Polar, Gaussian, Gaussian Right Focus, and Gaussian Right Polar are higher for a higher stage bladder cancer compared to a lower stage bladder cancer.

In some embodiments, a method of staging (determining stage or severity of) bladder cancer in the subject in need thereof includes obtaining or performing a magnetic resonance (MR) scan to obtain an image of a urothelial tissue or bladder of the subject, and measuring in a region of interest (ROI) comprising or consisting of a tumor in the urothelial tissue or the bladder one or more radiomic features of Energy, Cluster Prominence, Cluster Shade, Cluster Tendency (also known as Cluster Trend), Homogeneity, Autocorrelation, Inverse Gaussian Left, Inverse Gaussian Left Focus, Gaussian Right Polar, Gaussian, Gaussian Right Focus, and Gaussian Right Polar, wherein a higher level of the one or more radiomic features than respective level in a control subject with T1 or T2 bladder cancer or in a control subject with normal urothelium indicates that the subject in need thereof has a stage III (T3) or IV (T4) bladder cancer.

In some embodiments, a method of staging (determining stage or severity of) bladder cancer in the subject in need thereof includes measuring a higher level than control of each of the one, two, three, four, five, six, seven, eight, nine, ten, 11 or 12 radiomic features selected from the group consisting of Energy, Cluster Prominence, Cluster Shade, Cluster Tendency (also known as Cluster Trend), Homogeneity, Autocorrelation, Inverse Gaussian Left, Inverse Gaussian Left Focus, Gaussian Right Polar, Gaussian, Gaussian Right Focus, and Gaussian Right Polar, wherein the control is the radiomic features in an MR image of bladder cancer tissue of stage T1 or T2, thereby the subject in need there of being indicated to have a stage T3 or T4 bladder cancer. In further embodiments, a subject indicated to have a stage T3 or T4 bladder cancer is provided with a treatment for high-stage bladder cancer.

Various embodiments provide that a subject indicated, assayed, or diagnosed with a late/high stage bladder cancer such as T3 or T4, or N+ or M+, bladder cancer is provided with an aggressive treatment such as radiation therapy with concominant chemotherapy, radical cystectomy, which may follow, or be accompanied by, chemoradiotherapy (and anti-cancer drugs) and/or immunotherapy, or chemoradiotherapy (and anti-cancer drugs) and/or immunotherapy alone.

### Diagnosis & Treatment of Intra-Vesicle Bladder Cancer

In some embodiments, a method is provided for assaying a urothelial/bladder tissue in a subject in need thereof, e.g., for staging (determining stage or severity of) bladder cancer in the subject in need thereof, and optionally providing treatment therefor, which include measuring in a urothelial/bladder tissue sample an expression level of one or more of HOXB5, DHRS3, FABP4, TAGLN2, HIST1H1D, HIST1H2BD, H2AFX, CLDN3, and PLAUR, wherein (i) a higher expression level of HOXB5, DHRS3, and FABP4, each one if measured, (ii) a lower expression level of TAGLN2, HIST1H1D, HIST1H2BD, H2AFX, CLDN3, and PLAUR, each one if measured, or both (i) and (ii), compared to respective levels in a subject having a high stage bladder cancer such as T3 or T4, indicate that the subject in need thereof has an intra-vesicle bladder cancer (e.g., T1 or T2).

In some embodiments, an average being higher than a reference level of the (normalized) gene expression levels of all, or at least 95%, 90%, 85%, 80%, 75%, 70%, 60%, or 50%, of the genes in each one of the five, or any one, two, three, or four, of the Luminal gene set, the Luminal differentiation gene set, the Neuroendocrine differentiation gene set, the Normal Basal Intermediate gene set, and the Normal CDH12 gene set indicates that the subject has a stage I (T1) or II (T2) bladder cancer, or in some instances a normal urothelium, wherein the reference level is an average of respective (normalized) gene expression levels detected from a sample of a subject with high-stage (T3 or T4) bladder cancer or urothelial carcinoma. Urothelial carcinoma is a main type of bladder cancer, e.g., over 90% of bladder cancer is urothelial carcinoma, and remaining is squamous, adeno, small cell, micro papillary (variants). In some instances for a plurality of genes, a normalized expression level is used for each gene in order to compute an average, e.g., an average is the sum of each gene's normalized expression level divided by the number of genes. For example, for determining or indicating a intra-vesical bladder cancer, the expression level for a gene in a known normal urothelium is set as 100%, and the expression level for the gene in a tested sample is normalized against that in the normal urothelium sample.

In some embodiments, expression levels being higher than respective reference levels in all, or at least 95%, 90%, 85%, 80%, 75%, 70%, 60%, or 50%, of the genes in each one of the five, or any one, two, three, or four, of the Luminal gene set, the Luminal differentiation gene set, the Neuroendocrine differentiation gene set, the Normal Basal Intermediate gene set, and the Normal CDH12 gene set indicates that the subject has a stage I (T1) or II (T2) bladder cancer, or in some instances a normal urothelium, wherein reference level is that detected from a sample of a subject with extra-vesical (T3 or T4) bladder cancer.

In some embodiments, a subject indicated to have a stage I (T1) or II (T2) bladder cancer also has an average being lower than a reference level of the (normalized) gene expression levels of all, or at least 95%, 90%, 85%, 80%, 75%, 70%, 60%, or 50%, of the genes in each one of the ten, or any one, two, three, four, five, six, sever, eight, or nine, of the Basal gene set, the Basal Differentiation gene set, the Base Excision Repair gene set, the Cell Cycle gene set, the Homologous Recombination gene set, the MicroRNAs in Cancer gene set, the Oocyte Meiosis gene set, the p53 Signaling Pathway gene set, the Progesterone-mediated Oocyte Maturation gene set, and the Pyrimidine Metabolism gene set, wherein the reference level is an average detected from a sample of a subject with stage T3 or T4 bladder cancer. Hence, in some embodiments, an average being lower than a reference level of (normalilzed) gene expression levels of all, or at least 95%, 90%, 85%, 80%, 75%, 70%, 60%, or 50%, of the genes in each one of the ten, or any one, two, three, or four, five, six, sever, eight, or nine, of the Basal gene set, the Basal Differentiation gene set, the Base Excision Repair gene set, the Cell Cycle gene set, the Homologous Recombination gene set, the MicroRNAs in Cancer gene set, the Oocyte Meiosis gene set, the p53 Signaling Pathway gene set, the Progesterone-mediated Oocyte Maturation gene set, and the Pyrimidine Metabolism gene set indicates the subject has a stage I (T2) or II (T2) bladder cancer, wherein the reference level is an average detected from a sample of a subject with T3 or T4 bladder cancer.

In some embodiments, a subject indicated to have a stage I (T1) or II (T2) bladder cancer also has expression levels being lower than respective reference levels in all, or at least 95%, 90%, 85%, 80%, 75%, 70%, 60%, or 50%, of the genes in each one of the ten, or any one, two, three, or four, five, six, sever, eight, or nine, of the Basal gene set, the Basal Differentiation gene set, the Base Excision Repair gene set, the Cell Cycle gene set, the Homologous Recombination gene set, the MicroRNAs in Cancer gene set, the Oocyte Meiosis gene set, the p53 Signaling Pathway gene set, the Progesterone-mediated Oocyte Maturation gene set, and the Pyrimidine Metabolism gene set, wherein reference level is that detected from a sample of a subject with T3 or T4 bladder cancer. Hence, in some embodiments, expression levels being lower than respective reference levels in all, or at least 95%, 90%, 85%, 80%, 75%, 70%, 60%, or 50%, of the genes in each one of the ten, or any one, two, three, or four, five, six, sever, eight, or nine, of the Basal gene set, the Basal Differentiation gene set, the Base Excision Repair gene set, the Cell Cycle gene set, the Homologous Recombination gene set, the MicroRNAs in Cancer gene set, the Oocyte Meiosis gene set, the p53 Signaling Pathway gene set, the Progesterone-mediated Oocyte Maturation gene set, and the Pyrimidine Metabolism gene set, indicates that the subject has a stage I (T1) or II (T2) bladder cancer, wherein reference level is that detected from a sample of a subject with T3 or T4 bladder cancer.

In some embodiments, a method of staging (determining stage or severity of) bladder cancer in the subject in need thereof includes obtaining or performing a magnetic resonance (MR) scan to obtain an image of a urothelial tissue or bladder of the subject, and measuring in a region of interest (ROI) comprising or consisting of a tumor in the urothelial tissue or the bladder one or more radiomic features of Energy, Cluster Prominence, Cluster Shade, Cluster Tendency (also known as Cluster Trend), Homogeneity, Autocorrelation, Inverse Gaussian Left, Inverse Gaussian Left Focus, Gaussian Right Polar, Gaussian, Gaussian Right Focus, and Gaussian Right Polar, wherein a lower level of the one or more radiomic features than respective level in a control subject with T3 or T4 bladder cancer indicates that the subject in need thereof has a stage I (T1) or II (T2) bladder cancer.

Various embodiments provide that a subject indicated, assayed, or diagnosed with a intra-vesical bladder cancer such as T1 or T2 bladder cancer is provided with a treatment to preserve the bladder. Exemplary bladder preservation treatments against low stage cancer include transurethral resection of bladder tumor (TURBT) followed by intravesical Bacillus Calmette-Guerin (BCG) (introduced into bladder as a liquid through a catheter, as a type of intravesical (in bladder) immunotherapy) or chemotherapy (including anti-cancer drugs). In some embodiments, patients diagnosed with a T2 or greater bladder cancer may undergo further invasive surgery (radical cystectomy) with systemic chemotherapy before or after the surgery or immunotherapy after the surgery to remove cancerous tissue/cells.

Anticancer drugs include but are not limited to cisplatin, carboplatin, oxaliplatin, nedaplatin, triplatin tetranitrate, phenanthriplatin, picoplatin, satraplatin, taxane, anti-VEGF therapy, as well as other new treatments. Such other anti-cancer therapies will be expected to act in an additive or synergistic manner with the immune checkpoint blockade therapy.

Exemplary immunotherapeutics include but are not limited to immune checkpoint therapies such as antibody-based immune checkpoint therapy and immune checkpoint inhibitors. These include but are not limited to, an anti-PD-L1 antibody, an antibody against PD-1, an antibody against PD-L2, an antibody against CTLA-4, an antibody against KIR, an antibody against IDO1, an antibody against IDO2, an antibody against TIM-3, an antibody against LAG-3, an antibody against OX40R, and an antibody against PS.

Other examples of immune checkpoint inhibitors include inhibitors of leukocyte surface antigen CD47 (antigenic surface determinant protein OA3 or integrin associated protein or protein MER6 or CD47), and such examples are magrolimab (by Forty Seven), IBI-188 (by Innovent Biologics), ALX-148 (by ALX Oncology), AO-176 (by Arch Oncology), and CC-90002 (by Bristol-Myers Squibb).

Another class of exemplary immune checkpoint inhibitors or immune checkpoint blockade therapeutics include antagonists or inhibitors of T cell immunoreceptor with Ig and ITIM domains (V set and immunoglobulin domain containing protein 9 or V set and transmembrane domain containing protein 3 or TIGIT), and such examples are tiragolumab (by Genentech), AB-154 (by Arcus Biosciences), BMS-986207 (by Bristol-Myers Squibb), vibostolimab (by Merck), and BGBA-1217 (by BeiGene).

Yet another class of exemplary immune checkpoint inhibitors or immune checkpoint blockade therapeutics include antagonists of adenosine receptor A2a (ADORA2A) or A2b (ADORA2B), and examples include AB-928 (by Arcus Biosciences), ciforadenant (by Corvus Pharmaceuticals), HTL-1071 (by AstraZeneca), PBF-509 (by Novartis), and EOS-100850 (by iTeos Therapeutics).

In one embodiment, the immune checkpoint inhibitor is humanized monoclonal anti-programmed death ligand 1 (PD-L1) antibody, atezolizumab. In another embodiment, the immune checkpoint inhibitor is an anti-PD-L1 antibody such as avelumab, durvalumab, KN035, CK-301, AUNP12, CA-170, MPDL3280A (RG7446), MEDI4736 and BMS-936559.

In another embodiment, the immune checkpoint inhibitor is an anti-PD-1 antibody such as pembrolizumab (formerly lambrolizumab or MK-3475), nivolumab (BMS-936558), cemiplimab, spartalizumab, camrelizumab, sintilimab, tislelizumab, toripalimab, Pidilizumab (CT-011), AMP-224, or AMP-514.

Further examples of immune checkpoint inhibitor, or immune checkpoint blockade (ICB) therapeutics, include but are not limited to, B7-DC-Fc fusion proteins such as AMP-224, anti-CTLA-4 antibodies such as tremelimumab (CP-675,206) and ipilimumab (MDX-010), antibodies against the B7/CD28 receptor superfamily, anti-Indoleamine (2,3)-dioxygenase (IDO) antibodies, anti-IDO1 antibodies, anti-IDO2 antibodies, tryptophan, tryptophan mimetic, 1-methyl tryptophan (1-MT)), Indoximod (D-1-methyl tryptophan (D-1-MT)), L-1-methyl tryptophan (L-1-MT), TX-2274, hydroxyamidine inhibitors such as INCB024360, anti-TIM-3 antibodies, anti-LAG-3 antibodies such as BMS-986016, recombinant soluble LAG-3Ig fusion proteins that agonize MHC class II-driven dendritic cell activation such as IMP321, anti-KIR2DL1/2/3 or anti-KIR) antibodies such lirilumab (IPH2102), urelumab (BMS-663513), anti-phosphatidylserine (anti-PS) antibodies such as Bavituximab, anti-idiotype murine monoclonal antibodies against the human monoclonal antibody for N-glycolil-GM3 ganglioside such as Racotumomab (formerly known as 1E10), anti-OX40R antibodies such as IgG CD134 mAb, anti-B7-H3 antibodies such as MGA271, and small interfering (si) RNA-based cancer vaccines designed to treat cancer by silencing immune checkpoint genes.

In various embodiments, the immune checkpoint inhibitor is formulated into a pharmaceutical composition. Pharmaceutical compositions according to the invention may be formulated for delivery via any route of administration.

### Additional Embodiments

Methods of conducting examination of a subject in need thereof are also provided, which include i) measuring an expression level of one or more of genes HIST1H1D, H2AFX, PLAUR, TAGLN2, HIST1H2BD, CLDN3, DHRS3, HOXB5, and FABP4 in a biological sample of the subject; ii) performing radiomic analysis on a multi-parametric magnetic resonance imaging (MRI) image associated with the bladder of the subject, wherein the radiomic analysis comprises extracting from the MRI image a set of radiomic features of Energy, Cluster Prominence, Cluster Shade, Cluster Tendency, Homogeneity, Autocorrelation, Inverse Gaussian Left, Inverse Gaussian Left Focus, Gaussian Right Polar, Gaussian, Gaussian Right Focus, and Gaussian Right Polar; and/or iii) measuring expression levels of all or at least one gene in one or more of gene sets Basal, Basal Differentiation, Base Excision Repair, Cell Cycle, Homologous Recombination, MicroRNAs in Cancer, Oocyte Meiosis, p53 Signaling Pathway, Progesterone-mediated Oocyte Maturation, Pyrimidine Metabolism, Luminal, Luminal differentiation, Neuroendocrine differentiation, Normal Basal Intermediate, and Normal CDH12.

In some embodiments, the subject in need thereof is a human with suspected bladder tumor or human with a sessile appearing bladder mass.

In some embodiments, the biological sample comprises a bladder biopsy, a resected bladder tumor specimen, or a cystectomy specimen.

In some embodiments, the measuring of a gene expression level comprises performing single-cell RNA sequencing

Some embodiments provide methods for detecting a stage I (T1) or II (T2) bladder cancer in a subject, which include: detecting a higher expression level of HOXB5, DHRS3, and/or FABP4 in a biological sample of the subject relative to respective level in a reference subject with stage III (T3) or IV (T4) bladder cancer; detecting a lower expression level of TAGLN2, HIST1H1D, HIST1H2BD, H2AFX, CLDN3, and/or PLAUR in the biological sample relative to respective level in the reference subject with stage III (T3) or IV (T4) bladder cancer.

Some embodiments provide methods for detecting a stage I (T1) or II (T2) bladder cancer in a subject, which include performing radiomic analysis on a multi-parametric magnetic resonance imaging (MRI) image associated with the bladder of the subject and detecting a lower level of radiomic features relative to respective level in a reference subject with stage III (T3) or IV (T4) bladder cancer, wherein the radiomic features include one or more of Energy, Cluster Prominence, Cluster Shade, Cluster Tendency, Homogeneity, Autocorrelation, Inverse Gaussian Left, Inverse Gaussian Left Focus, Gaussian Right Polar, Gaussian, Gaussian Right Focus, and Gaussian Right Polar.

Some embodiments provide methods for detecting a stage I (T1) or II (T2) bladder cancer in a subject, which include detecting a lower gene expression of all or at least one gene in one or more gene sets of Basal, Basal Differentiation, Base Excision Repair, Cell Cycle, Homologous Recombination, MicroRNAs in Cancer, Oocyte Meiosis, p53 Signaling Pathway, Progesterone-mediated Oocyte Maturation, Pyrimidine Metabolism, relative to respective levels in a reference subject with stage III (T3) or IV (T4) bladder cancer; and/or detecting a higher gene expression of all or at least one gene in one or more gene sets of Luminal, Luminal differentiation, Neuroendocrine differentiation, Normal Basal Intermediate, and Normal CDH12.

Further embodiments of detecting a stage I (T1) or II (T2) bladder cancer include identifying two or more of the indicators above.

Additional embodiments provide methods for detecting a stage III (T3) or IV (T4) bladder cancer in a subject, which include detecting a lower expression level of HOXB5, DHRS3, and/or FABP4 in a biological sample of the subject relative to respective level in a reference subject with stage I (T1) or stage II (T2) bladder cancer; detecting a higher expression level of TAGLN2, HIST1H1D, HIST1H2BD, H2AFX, CLDN3, and/or PLAUR in the biological sample relative to respective level in the reference subject with stage I (T1) or II (T2) bladder cancer.

Some embodiments provide methods for detecting a stage III (T3) or IV (T4) bladder cancer in a subject, which include performing radiomic analysis on a multi-parametric magnetic resonance imaging (MRI) image associated with the bladder of the subject and detecting a higher level of radiomic features relative to respective level in a reference subject with stage I (T1) or II (T2) bladder cancer, wherein the radiomic features include one or more of Energy, Cluster Prominence, Cluster Shade, Cluster Tendency, Homogeneity, Autocorrelation, Inverse Gaussian Left, Inverse Gaussian Left Focus, Gaussian Right Polar, Gaussian, Gaussian Right Focus, and Gaussian Right Polar.

Some embodiments provide methods for detecting a stage III (T3) or IV (T4) bladder cancer in a subject, which include detecting a higher gene expression of all or at least one gene in one or more gene sets of Basal, Basal Differentiation, Base Excision Repair, Cell Cycle, Homologous Recombination, MicroRNAs in Cancer, Oocyte Meiosis, p53 Signaling Pathway, Progesterone-mediated Oocyte Maturation, Pyrimidine Metabolism, relative to respective levels in a reference subject with stage I (T1) or II (T2) bladder cancer; and/or detecting a lower gene expression of all or at least one gene in one or more gene sets of Luminal, Luminal differentiation, Neuroendocrine differentiation, Normal Basal Intermediate, and Normal CDH12.

Further embodiments of detecting a stage III (T3) or IV (T4) bladder cancer include identifying two or more of the indicators above.

### EXAMPLES

The following examples are provided to better illustrate the claimed invention and are not to be interpreted as limiting the scope of the invention. To the extent that specific materials are mentioned, it is merely for purposes of illustration and is not intended to limit the invention. One skilled in the art may develop equivalent means or reactants without the exercise of inventive capacity and without departing from the scope of the invention.

### Example 1

In this first study, an extensive radiogenomic analysis of MIBC cases was performed to identify potential indicators of MIBC stages and to automate the staging process. A total of 28 MRI scans, and their matched transcriptomic profiles were obtained from subjects diagnosed with MIBC at different stages. This is a “Discovery” cohort, also further divided as 75% “training” dataset and 25% “testing” dataset, performed in Kyoto University Hospital for 28 cases (MRIs and RNA-seq), in which 18 cases were bladder tumors that are clinical intra-vesical (T0, T1, T2) and 10 other cases were bladder tumors that are clinical extra-vesical (T3, T4).

Additionally, an external validation cohort would be performed in Nara Medical University Hospital for 12 cases (MRIs and RNA-seq), in which 6 cases were bladder tumor that are clinical intra-vesical (T0, T1, T2) and 6 other cases were tumor clinical extra-vesical (T3, T4).

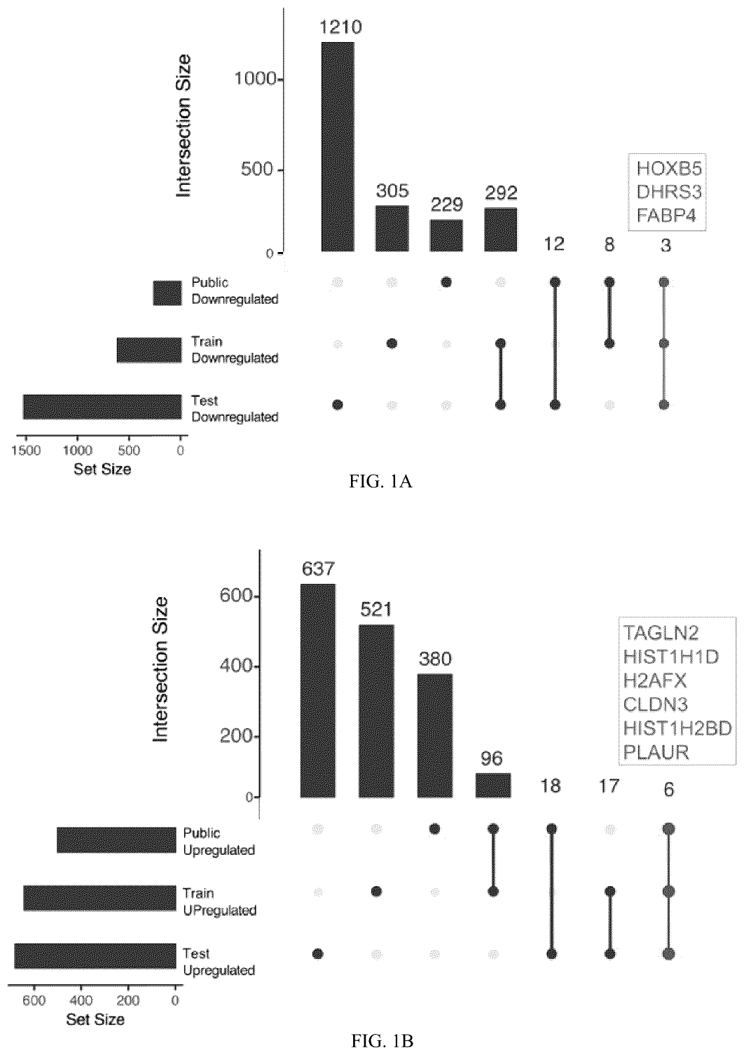

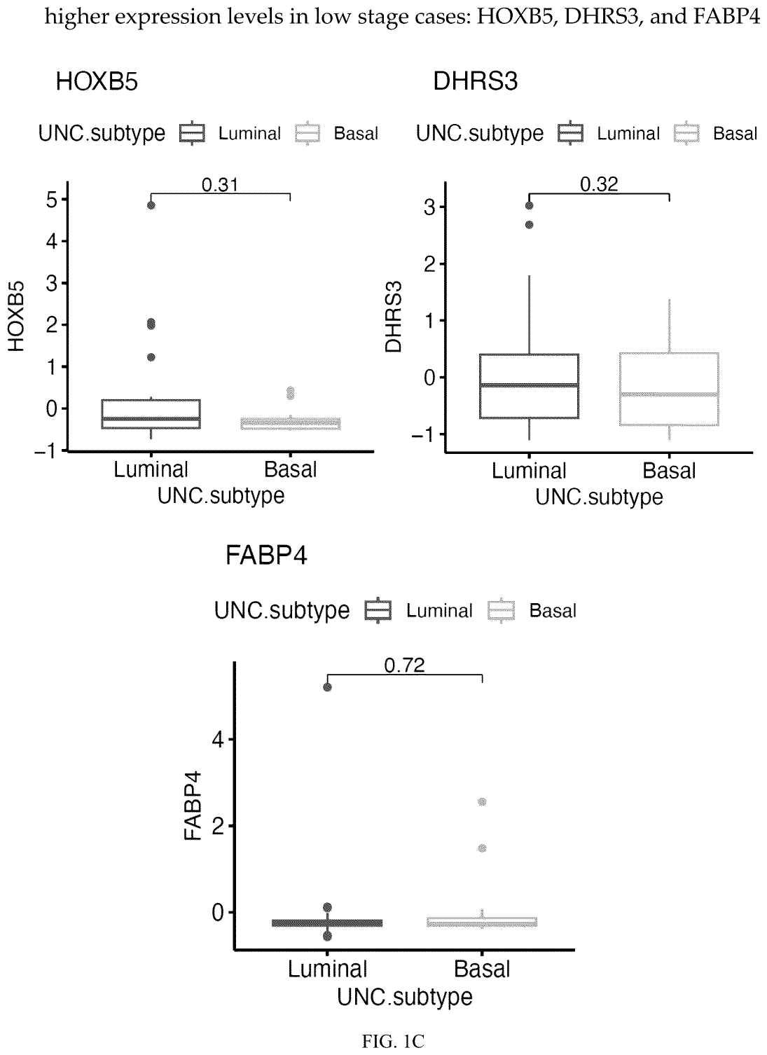

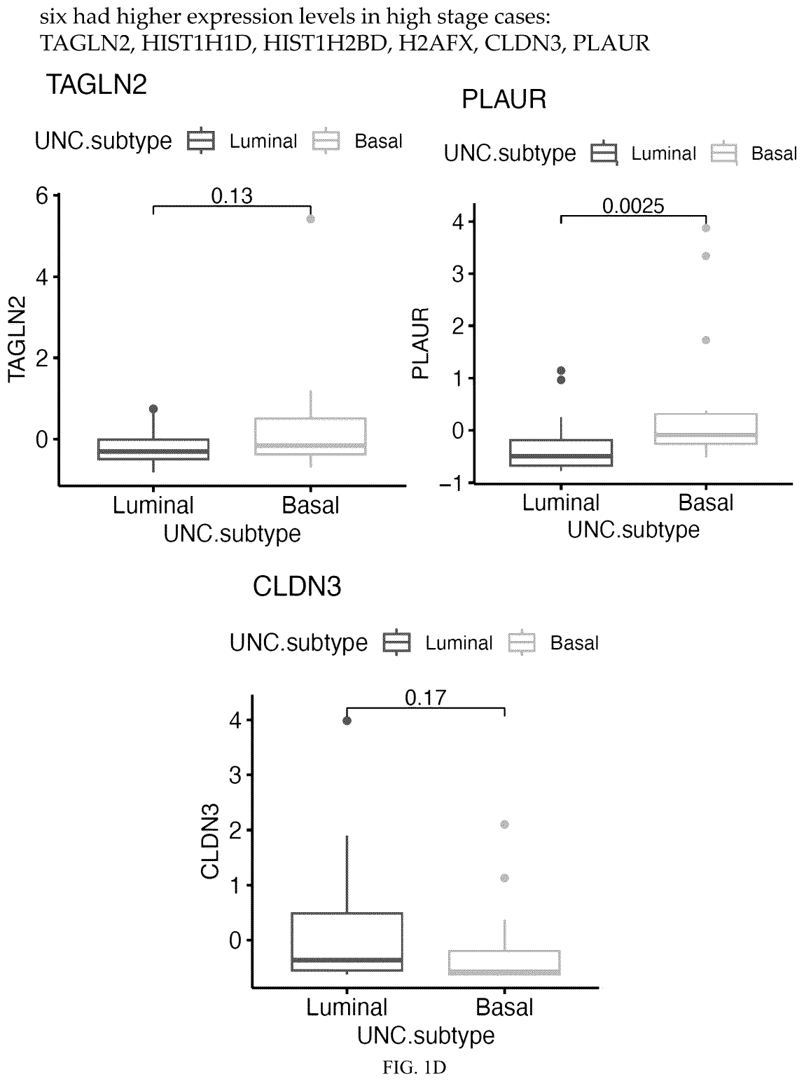

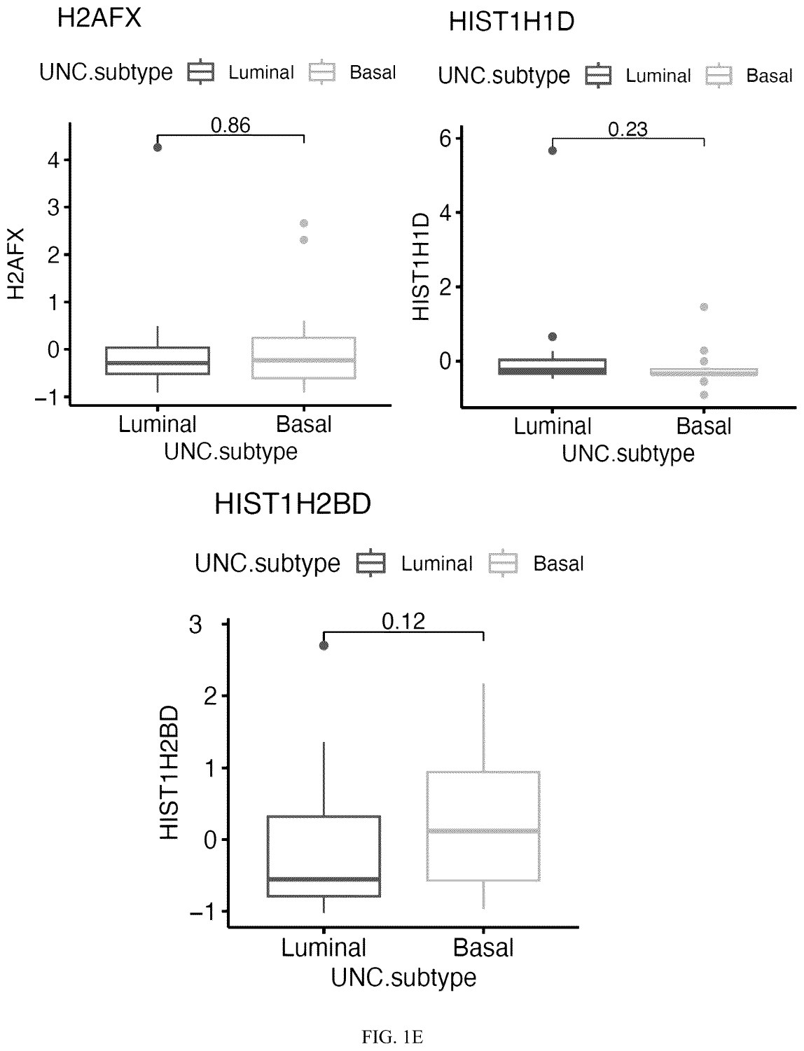

Additionally, “Public” genomics data was obtained from two cohorts: Cohort 1 was from Liu Y. et a., European Urology, vol. 66, issue 6, December 2014, pages 982-986, which has high stage (n=22) and low stage (n=27) RNA-seq data; and Cohort 2 was from Bondaruk J. et al., iScience 25, 104551, Jul. 15, 2022, which has whole-organ histologic and genomic mapping of 9 patients (e.g., see Bondaruk's FIG. 1, panels D-G). For analytical purposes, samples of mild dysplasia (MD) and moderate dysplasia (MdD) were combined and referred to as low-stage intraurothelial neoplasia (LGIN), and samples of severe dysplasia (SD) and carcinoma in situ (CIS) were combined and referred to as high-stage intraurothelial neoplasia (HGIN).

In the genomics, a bulk RNA-seq from formalin-fixed paraffin-embedded (FFPE) tissues was performed that identified 3 major clusters: high, intermediate, and low infiltrating cells including immune cells and fibroblasts. This was followed by a bioinformatic analyses, such as pathway enrichment and gene set enrichment analyses, 15 most significant signatures were identified for stage categorization of MIBC.

Specifically, for genomics features selection: we collected 72 bladder cancer (BLCA)-stage-related signatures from the published references and applied the ssGSEA algorithm to our training and test datasets, as well as two published cohorts.

TABLE 1
Referenced patient database.

| | | MIBC | Stage |
|---|---|---|---|
| Dataset | Publication | samples | (T2/T3/T4) |
| Dyrskjot | Cancer Res. 2004 | 31 | 3/12/15 |
| Sanchez | J. Clin. Oncol. 2006 | 53 | 9/34/10 |
| Stransky | Nat. Genet. 2006 | 39 | 9/19/11 |
| Kim | Mol. Med. 2011 | 61 | 31/19/11 |
| Riester | Clin. Cancer Res. 2012 | 78 | 17/42/19 |
| Sjödahl2012 | Clin. Cancer Res. 2012 | 93 | 85/7/1 |
| Choi MDA discovery | Cancer Cell 2014 | 73 | 42/23/8 |
| Choi MDA MVAC | Cancer Cell 2014 | 23 | 15/7/1 |
| Choi MDA validation | Cancer Cell 2014 | 46 | 39/7/0 |
| Choi Philadelphia | Cancer Cell 2014 | 42 | 16/19/7 |
| NAC | | | |
| CIT | Sci. Transl. Med. 2014 | 87 | 31/37/19 |
| Seiler | Eur. Urol. 2017 | 305 | 143/109/49 |
| Sjödahl2017 | J. Pathol. 2017 | 243 | 241/1/1 |
| TCGA | Cell 2017 | 406 | 122/194/58 |
| Our SnRNA-seq data | Nature communications | 24 | 21/2/1 |
| | 2020 | | |

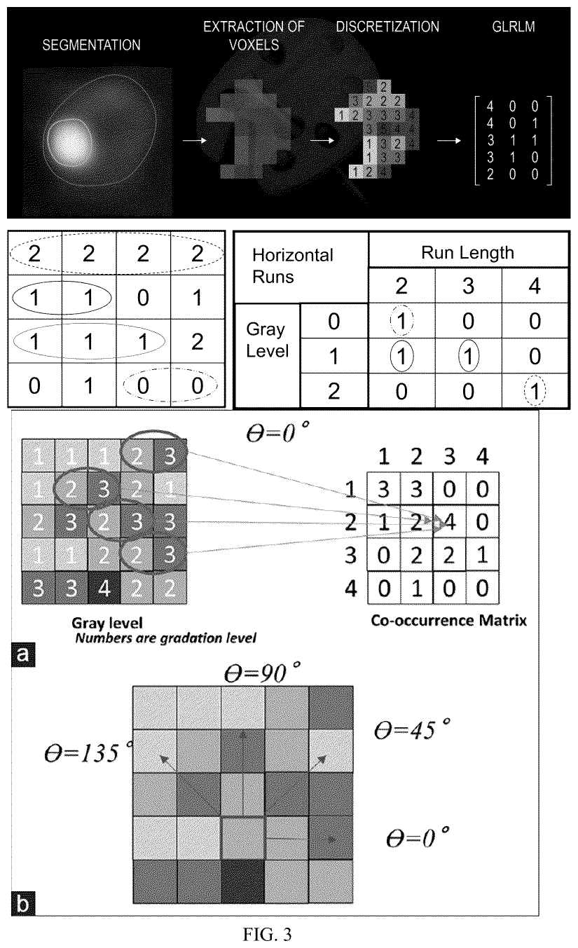

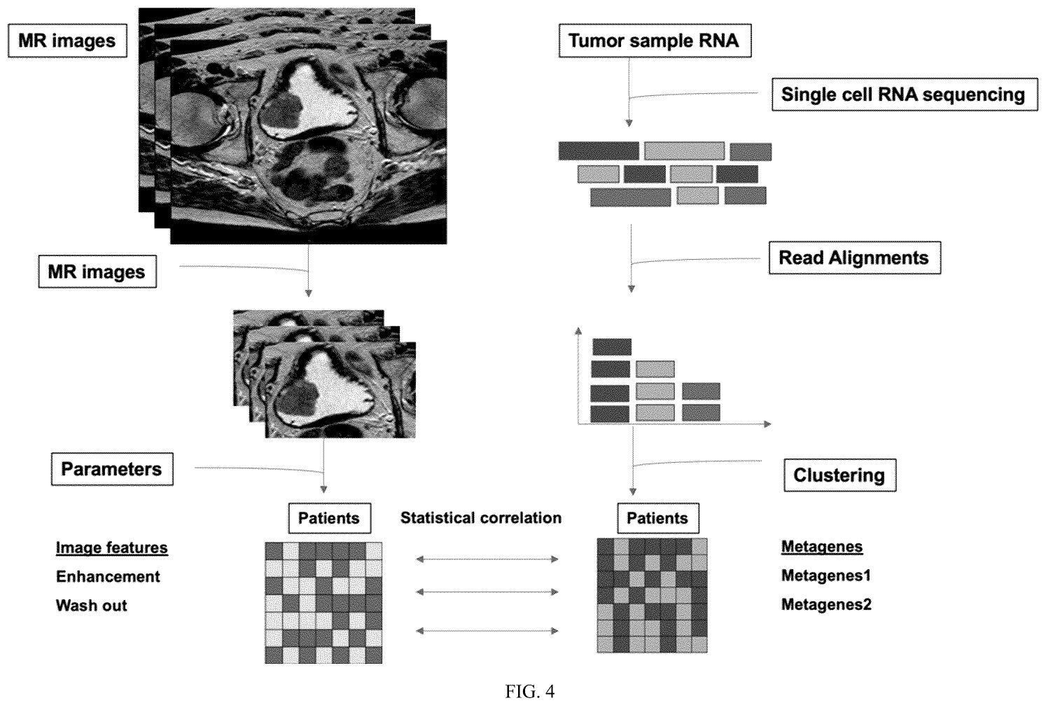

In the radiomics, several hundred image-based features (mainly based on signal intensity, texture, size, and shape of tumor) from bladder tumor in MRI were extracted and analyzed. The tumors were manually outlined and grouped into intra-vesical (stage I and II) and extra-vesical (stage III and IV) by the trained radiologists. Using various statistical tests, several radiomic features (e.g., heterogeneity, inverse contrast, entropy) were inferred as significant at discriminating the two groups (FIG. 3). In some implementations, the lower-stage further includes stage 0.

A naïve Bayes machine learning model was then used to integrate such features and perform automated binary classification of cases into their true class (i.e., lower stage or higher stage). A 4-fold cross validation was performed in which a unique 75% and 25% of 28 cases were used for training and testing of the model respectively.

Results: The model performance was assessed in terms of prediction (binary classification) of lower-stage vs higher-stage as mean sensitivity, specificity, and accuracy, reaching 84%, 86%, and 86% respectively. The radiogenomics of MIBC provides insight to several features associated with cancer stages. The discriminative features help accurately staging MIBC.

Therefore, the radiogenomics of MIBC provides insight to several features associated with cancer stages. Assisted with AI, the discriminative features help accurately staging MIBC.

### Example 2

We aimed to develop a reliable radiogenomic assay to accurately stratify patients with muscle invasive bladder cancer (MIBC) according to their survival (progression free and overall), and this radiogenomic assay would supplant traditional computed tomography (CT) scan and lead to improved outcomes.

Multiparametric magnetic resonance imaging (mpMRI) is emerging as the imaging modality of choice in tumor staging, with a reported sensitivity of 90% in detecting BCa and 76% sensitivity with 89% specificity of detecting metastatic lymph nodes (2). The superior soft tissue contrast and versatile imaging sequences of MRI can facilitate margin definitions critical in cystectomy and bladder preservation strategies. In addition, 4D-MRI techniques have provided high isotropic resolution that was previously unachievable with standard MRI. With these images, extensive quantitative imaging metrics can be collected.

Recently, radiomics, defined as the high-throughput extraction of quantitative imaging metrics, has emerged as a potential diagnostic, capable of assessing the tumor's aggressiveness and extent; so as to garner a better tumor phenotype and predict clinical outcomes. Thus, 4D-mpMRI offers an opportunity to reduce staging errors through better anatomical visualization compared to traditional CT imaging. Imaging parameters with strong associations to genomic data may serve as reliable markers for tumor diagnosis (i.e., staging), prognosis and treatment response. In addition, integrating radiogenomic data may a) allow the exploration of molecular interconnections that explain various tumor features captured by the radiologic parameters and b) validate the mechanistic pathways supporting such interconnections, and thereby, allowing us to gain a deeper understanding of the cancer and its pathophysiology.

Genomics has long been the mechanism of assessing a tumor's clinical characteristics. With the advent of early versions of gene expression profiling and RNA sequencing, there has been a large amount of data available for assessing tumor morphology, molecular structure, and behavior. Furthermore, TCGA has reported clustering of mRNA expression converged into distinct subsets with differential epithelial-mesenchymal transition status (e.g., basal, luminal and neuroendocrine), which have been linked to outcomes and survival.

A previous study using single cell RNA sequencing (scRNA-seq) demonstrated transcriptional heterogeneity of bladder cancer, indicating that the bulk RNA-seq, similar to what is in TCGA, may have limitations in BCa. In particular, scRNA-seq of BCa revealed an interesting transcriptome, a platinum resistance gene, COX7B, and its surrogate marker, CD63, which was previously not identified. Consequently, employing scRNA-seq of MIBC, we have identified unique gene clusters including but not limited to EGFR, NRG1, NRXN1, PTPRC, VWF, COL6A2, CD247, and ITGAX.

We aimed to correlate radiomics findings with that of genetic signature in order to identify a radiogenomic profile of MIBC which could be used to accurately stage patients. Our central hypothesis is that a radiogenomic profile exists that a) is specifically associated with BCa and b) can be utilized to more effectively and accurately stage BCa.

We sought to develop a computational algorithm that predicts, from radiographic imaging data and genetic alterations from scRNA-seq data of a tumors, the relative proportions of predefined cancer cell populations, which lends to aggressiveness and BCa extent.

Specifically, we would apply 4D mpMRI with diffusion weighted imaging to significantly improve upon the resolution of imaging the bladder and pelvis. We would employ scRNA-seq to offer a ‘holistic’ view of MIBC (i.e., genetic alterations and cellular composition of tumor microenvironment). We would use a in-house unsupervised method to deconvolute dynamic imaging series as well as deconvolution and coupled clustering from bulk and single-cell genomics data.

There are approximately 1600 imaging parameters that are acquired from a standard MRI examination. These 1600 parameters assess contrast-enhancing tumor volumes, necrotic tumor volumes, hyperintense tumor volumes on FLAIR images (“non-enhancing and edema” regions, excluding voxels within the contrast-enhancing and necrotic segmentation), complete tumor volumes (contrast enhancing+non-enhancing and edema+necrotic tumor volumes, subsequently referred to as “total” volumes), quantification of tumor volumes, volume ratios, histogram quantification of Gaussian-normalized ADC (nADC), Gaussian-normalized relative blood flow and volume are just some of these parameters. Using an in-house code, over 500 relevant parameters will be applied and their utility confirmed in segmented images of a selected number of MRI examinations and correlated with final TNM tumor stage as well as outcome using a deep learning classifier, so as to reduce these 500+ parameters to a much smaller number for further validation.

We would then obtain and integrate the RNA-seq data with the MRI data (e.g., texture, shape and size) from the same patients to find associations between some hallmark MIBC differentially expressed genes and radiomic metrics. We would apply our deconvolution algorithm to tumors for which there is both RNA-seq and imaging data, and then identify features in the latter that predict the presence of specific tumor cell populations as well as predicts tumor aggressiveness and extent (i.e., stage). Alternatively, we could correlate imaging metrics with miRNA and other genomic parameters (e.g., lncRNA, DNA mutations, and copy number variations).

Previously, we reported a new 4D-MRI sequence based on 3D-radial sampling and slab-selective excitation in 10 consecutive patients with borderline respectable pancreatic cancer (22). The non-contrast 4D-MRI images showed significantly better contrast to noise ratio for the vessels that limit tumor respectability compared to 4D-CT with delayed contrast. Noting that potential radiomic imaging biomarkers must be reproducible, robust, and repeatable, we conducted a pilot study using our texture phantom to evaluate the performance of a panel of radiomic parameters on routine abdominal imaging. The phantom was scanned on 2 separate 3 T (Biograph mMR, Siemens Healthineers. USA). We assessed the repeatability and robustness on both scanners and the reproducibility by comparing the inter-scanner differences in the robustness and repeatability. Our results indicate that the reliability of radiomics parameters is dependent on the scanner and scanning settings. FFT and histogram analysis metrics show favorable performance (<5% variability) and hence reliability, at least across the 2 scanners in consideration. Thus, we believe the 4D-MRI has great potential in accurately defining organ anatomy, extent of disease and enhance readout of identified parameters. It is possible that additional maneuvers, empty/full bladder, contrast in bladder, etc. may be needed to better visualize the bladder and its surrounding structures more precisely.

Building upon this, we proposed to incorporate the new 4D-MRI sequencing with mpMRI and T2-weighted (T2W) image, diffusion-weighted imaging (DWI), and dynamic contrast-enhanced image (DCE) in bladder cancer patients. All images can include the whole pelvis, bladder, proximal urethra, and prostate (in males) or uterus, ovaries, fallopian tubes, and vagina (in females). Spin-echo T1-weighted (T1W) image and T2W images pre and post contrast will be acquired. Then using the validated reduced number of radiomics parameters from the above studies, we would apply these parameters to the MRI image files into new deep learning classifier to correlate with final pathologic TNM stage and outcome. After imaging, the subjects will undergo standard TURBT followed by cystectomy for histologic evaluation noting histology, grade and more importantly tumor stage. In addition, fresh frozen tumor, urine and blood will be collected and stored in the Biorepository for subsequent analysis.

To interrogate intratumoral heterogeneity systematically, we will isolate nuclei from tumors from these 10 patients. scRNA-seq libraries will be generated using the 10× Genomics Chromium platform, a droplet-based single-cell sequencing method, and will be sequenced on a NovaSeq system (Illumina). While inter-tumoral correlations of the single-cell transcriptional profiles will be lower than intra-tumoral correlations, the intra-tumoral correlations should show a broad spread (R˜0.2-0.7), consistent with intra-tumoral heterogeneity. Additionally, this heterogeneity can be verified by the presence of distinct intra-tumoral clusters of cells based on Leiden clustering of the single-cell transcriptional profiles.

Querying a small population for numerous parameters may lead to high false positive rates. Thus we will apply a sufficient number of radiomics parameters and genes in our network analyses; and prioritization of genes and their interactions will be done using Lynx, PINTA and String algorithms to perform annotation, clustering, enrichment analysis, and prediction of high-confidence triggers and mediators in various cell populations of interest. Predictions of gene interactions and reconstruction of molecular pathways for the phenotypes of interest will be done using PINTA and STRING algorithms. These pipelines will be executed at scale using cloud resources. The results will be annotated with information from Lynx knowledge base integrating >35 biological databases. Biostatistical and bioinformatic comparisons of the molecular profiles with MRI imaging characteristics will be performed using a variety of publicly available and locally developed software applications.

Pearson's Correlation hypothesis tests with 5% significance level (two-sided) and 80% power allows sample sizes of 10, 19, or 23 patients to detect a difference between the null hypothesis correlation of zero and the alternative hypothesis correlation of 0.76, 0.59, or 0.55, respectively. In addition, sensitivity and specificity of several potential predictors will be estimated. The maximum width of the 95% confidence interval for proportions (sensitivities or specificities) associated with sample sizes of 10, 19, or 23 patients are 0.63, 0.47, 0.43, respectively.

### Example 3. MRI/RNA-Seq-Based Radiogenomics and Artificial Intelligence for More Accurate Staging of Muscle-Invasive Bladder Cancer

Accurate staging of bladder cancer assists in identifying optimal treatment (e.g., transurethral resection vs. radical cystectomy vs. bladder preservation). However, currently, about one-third of patients are over-staged and one-third are under-staged. There is a pressing need for a more accurate staging modality to evaluate patients with bladder cancer to assist clinical decision-making. We hypothesize that MRI/RNA-seq-based radiogenomics and artificial intelligence can more accurately stage bladder cancer. A total of 40 magnetic resonance imaging (MRI) and matched formalin-fixed paraffin-embedded (FFPE) tissues were available for analysis. Twenty-eight (28) MRI and their matched FFPE tissues were available for training analysis, and 12 matched MRI and FFPE tissues were used for validation. FFPE samples were subjected to bulk RNA-seq, followed by bioinformatics analysis. In the radiomics, several hundred image-based features from bladder tumors in MRI were extracted and analyzed. Overall, the model obtained mean sensitivity, specificity, and accuracy of 94%, 88%, and 92%, respectively, in differentiating intra-vs. extra-bladder cancer. The proposed model demonstrated improvement in the three matrices by 17%, 33%, and 25% and 17%, 16%, and 17% as compared to the genetic- and radiomic-based models alone, respectively. The radiogenomics of bladder cancer provides insight into discriminative features capable of more accurately staging bladder cancer.

Bladder cancer is the fourth and twelfth most common cancer in men and women, respectively, in the United States. An estimated 83,190 newly diagnosed cases of bladder cancer and 16,840 deaths from bladder cancer are expected to occur in 2024. Bladder cancer has one of the highest recurrence rates of any tumor type. When diagnosed early as a T0/T1 lesion or even a T2 lesion, a cure with surgical resection is possible in a high percentage of cases, with a 5-year survival rate of >94% and >50%, respectively. However, once the tumor extends beyond the muscle lining of the bladder, the 5-year survival rate is <50%, while metastatic disease is almost always fatal, with an estimated median survival of 12 to 14 months and a 5-year survival of <20%. Muscle-invasive bladder cancer (MIBC) comprises approximately one-third of bladder cancer and is associated with significant morbidity and mortality. The treatment of patients with MIBC has traditionally been managed through radical cystectomy. However, radical cystectomy is accompanied by significant treatment morbidity and mortality, as well as a substantive change in the quality of life associated with the removal of the urinary bladder. On the other hand, for highly select patients, the outcomes of bladder preservation with chemoradiotherapy may offer comparable survival outcomes to those of radical cystectomy. Therefore, it is important to properly select the patients suitable for the bladder-preserving strategy. Currently, a robust staging modality (i.e., determining the extent of cancer) is lacking, with approximately one-third of patients being under-staged and one-third of patients being over-staged with axial imaging of the abdomen and pelvis with computed tomography (CT) imaging. Thus, accurate staging of bladder cancer is essential to identifying the optimal treatment.

In-depth investigation of genetic and tumor-imaging information associated with different stages of bladder cancer may provide insight into unique patterns that could efficiently assist in distinguishing early-stage (i.e., intra-vesical) from late-stage (i.e., extra-vesical) cancers. Validating reliable biomarkers for MIBC staging remains an ongoing challenge, as bladder cancer is known for its molecular and clinical heterogeneity, posing challenges in developing a universally applicable staging system. Clinical and pathological staging through transurethral resection of bladder tumors (TURBT) may be prone to inter-observer variability, thereby potentially affecting the consistency of staging results. Incorporating imaging modalities and genomic profiling can be advantageous.

Artificial intelligence offers numerous tools and techniques to thoroughly examine genomic and imaging data, as well as the integration of the genomic and imaging data with the expressed purpose of improving the accuracy of current staging. In recent years, a range of automated analysis and modeling techniques have been developed, particularly for genomic and radiomic data, including tools for extracting precise measurements of biomarkers and organs, unveiling complex features, and quantifying tissue characteristics. The advancements in radiomic, machine learning, and deep learning approaches have tremendously scaled up AI-based cancer management in several aspects. Taken together, we conceive that the state-of-the-art processing techniques and analysis tools associated with radiogenomics can more accurately stage bladder cancers. In this study, we (1) identified genetic signatures that significantly help characterize the stages of bladder cancer, (2) analyzed the morphological and textural properties of the bladder tumors in magnetic resonance (MR) scans to seek out unique features that are not appreciated by the human eye but can potentially assist in identifying the stage of cancer, and (3) developed an automated system that integrates both genetic and MR features and characterizes the bladder cancer as intra-vesical (T1 and T2), i.e., tumors still stay within the bladder, vs. extra-vesical (T3 and T4), i.e., tumors have grown through the muscle layer of the bladder and into the layer of fatty tissue.

### Identification of Genomics Features

The analysis revealed nine common genes, three of which exhibited higher expression levels in low-stage cases (lower expression levels in high stage): HOXB5, DHRS3, and FABP4, whereas six had higher expression levels in high-stage cases (lower expression levels in low stage): TAGLN2, HIST1H1D, HIST1H2BD, H2AFX, CLDN3, and PLAUR, as shown in FIGS. 1A-1B.

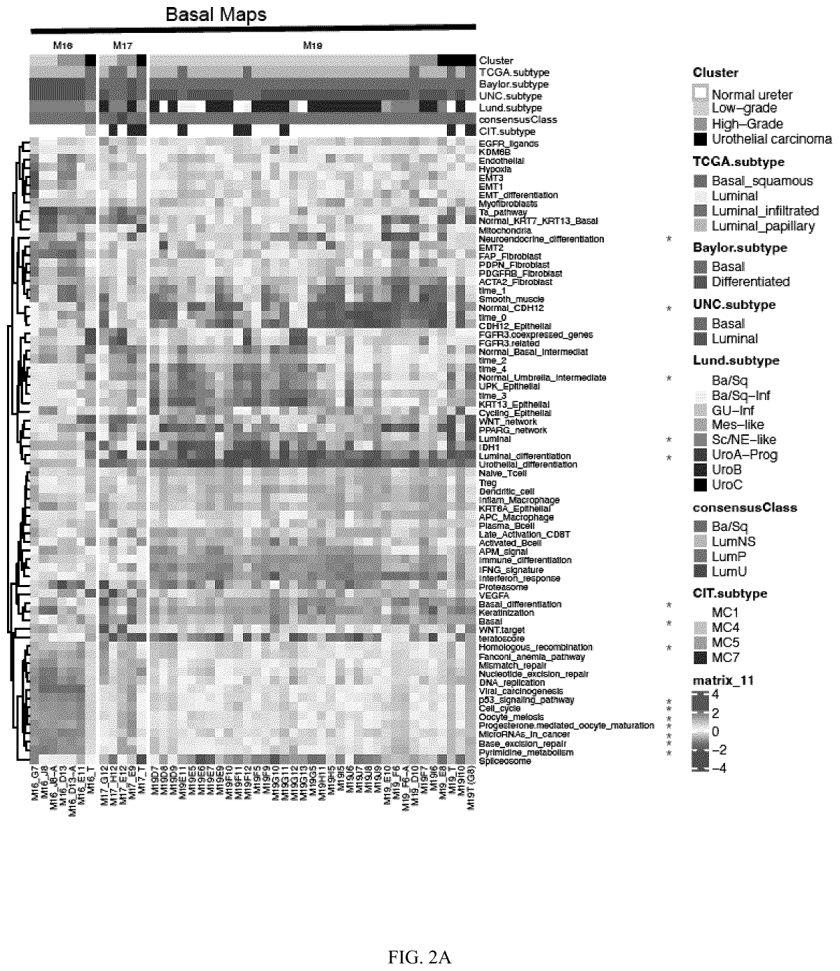

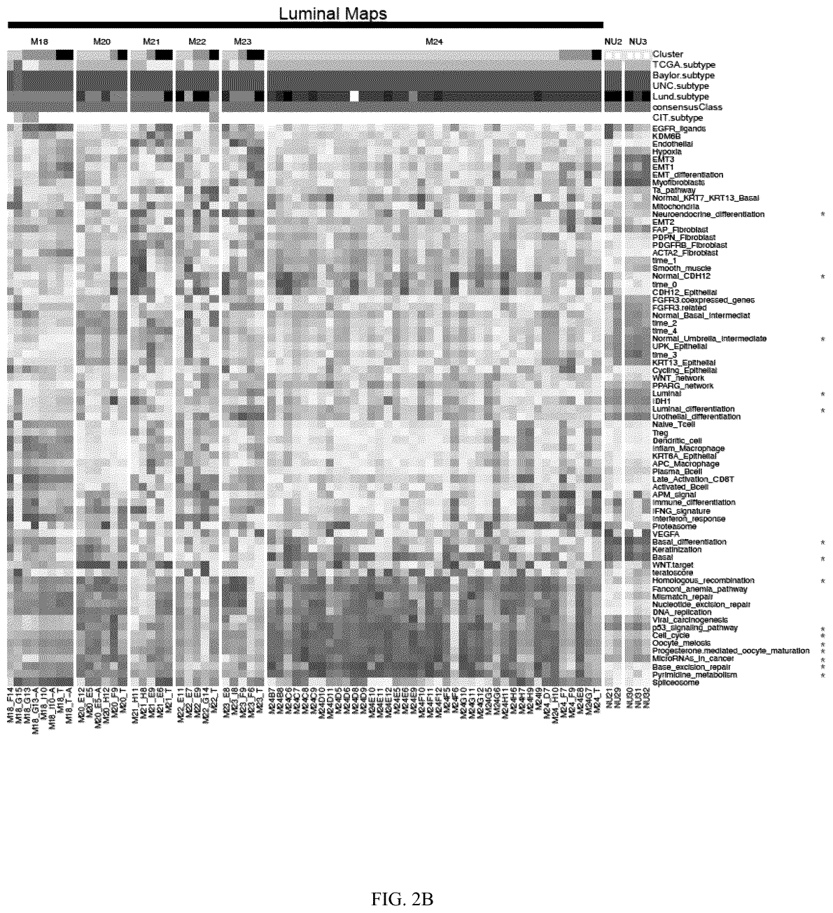

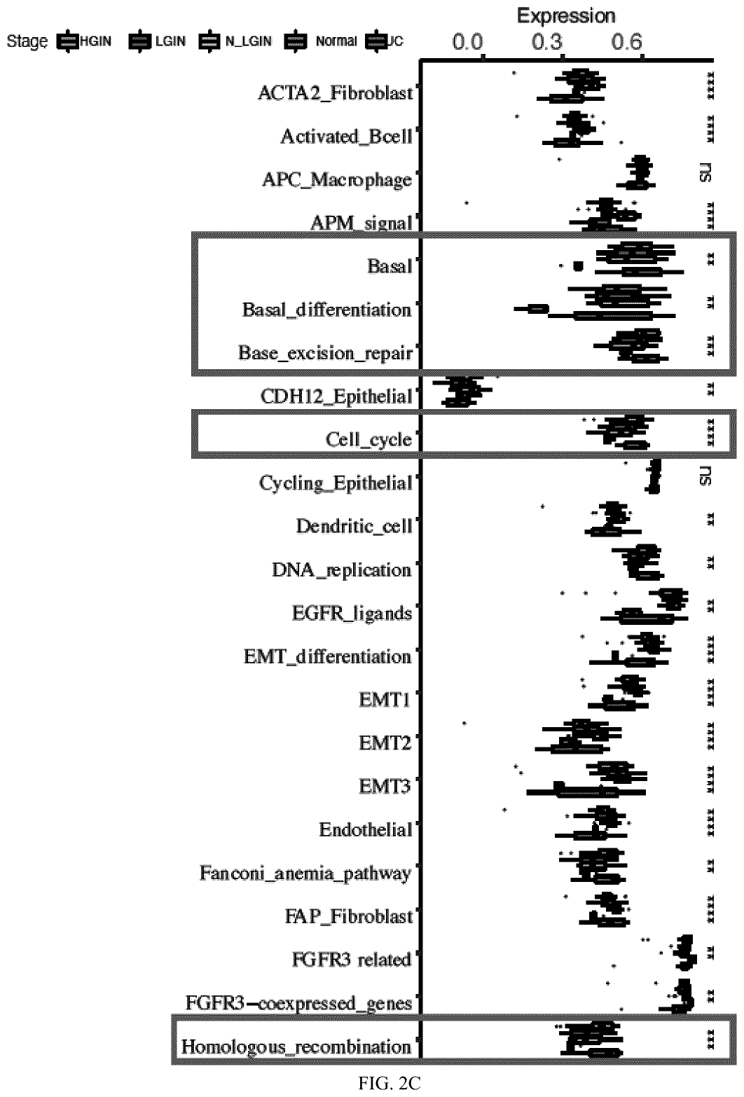

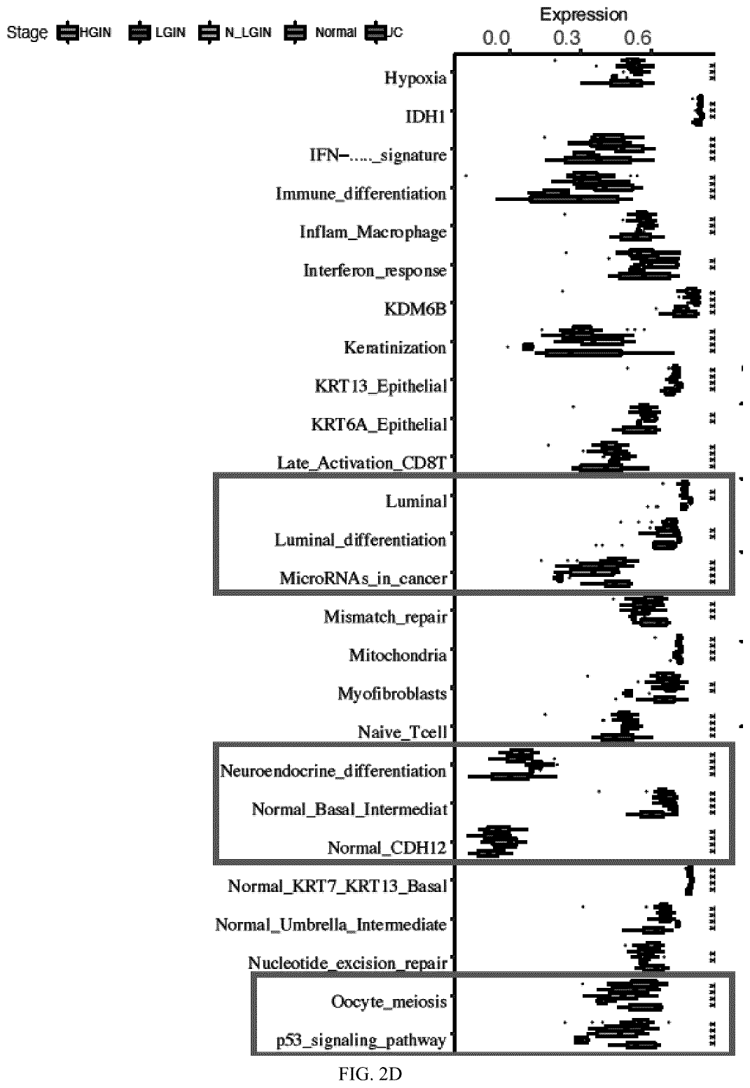

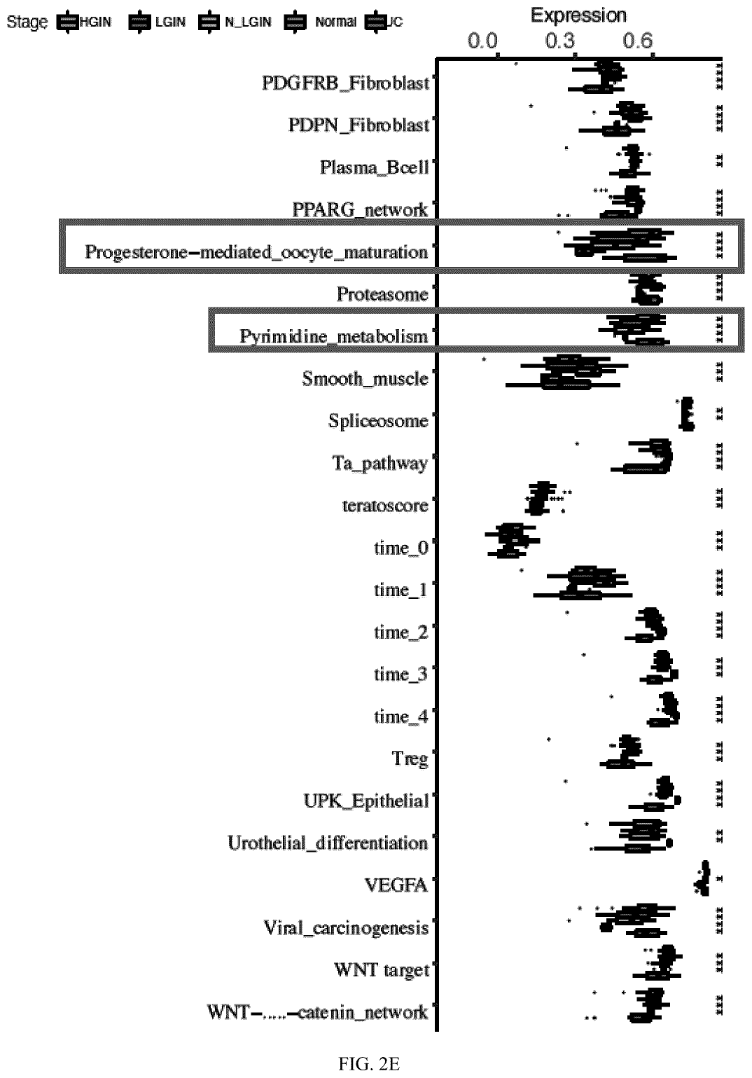

Based on the analysis using the single-sample Gene Set Enrichment Analysis (ssGSEA) algorithm, we successfully identified and corroborated 15 significant signatures related to the various stages of bladder cancer (as shown in FIG. 2C-2E). Among the identified signatures, some exhibited a decreasing trend from NU (normal urothelium) to UC (urothelial carcinoma), with the highest expression in normal urothelium and progressively decreasing through mild, moderate, and severe dysplasia stages and carcinoma in situ. These signatures were Luminal, Luminal differentiation, Neuroendocrine differentiation, Normal Basal Intermediate, and Normal CDH12. In contrast, other signatures demonstrated the opposite trend, being lowest in normal urotheliumas and increasing as dysplasia severity increased, reaching their peak in urothelial carcinoma, as given in FIG. 2C-2E. The results from these analyses were subsequently confirmed using comprehensive histological and genomic mapping data from two patients (one basal and one luminal). The findings obtained using Data_{K} were validated using Data_{W} and Data_{B}. The statistical significance test was performed on Data_{W} and Data_{B}, where the identified 9 and 15 genes were found to be significantly different in intra-vs. extra-vesical bladder cancer. We looked at the extent of the cancer using 3 variables: Extent of the primary tumor (T), Extent of tumor in lymph nodes (N), and Extent of tumor beyond lymph nodes or metastasis (M). T4 is the highest when looking at the primary tumor, but MI is the highest overall as it denotes widespread metastasis.

### Identification of Radiomics Features

Several statistical tests were performed on the extracted features from all 28 images of Data_{K} using different analysis tools, including R, Matlab, and SPSS. Three major tasks were completed in the following order: (1) obtain a set of clean and uniform features (e.g., by eliminating nonzero or inoperable values), (2) reduce the redundancy of the feature sets, and (3) identify the most significant features in terms of the statistical difference between extra- and intra-vesical tumors. The statistical Student's t-test and Bhattacharya coefficient were performed on the filtered set of features to identify the significantly different features between the two groups. About 33% of the total number of extracted radiomic features showed significance at a p-value of 0.05. The findings supported the primary hypothesis, as the analysis identified MR features distinguishing extra-vesical and intra-vesical bladder tumors. Most of the identified significant features were texture-based.

### Integration of Radiomics and Genomics Features Improves Bladder Cancer Staging

The performance of the proposed radiogenomic model for automated bladder cancer staging was evaluated by considering the staging as a binary classification problem where one MIBC case categorized as a false stage is considered a failure. The model was evaluated using three matrices, including sensitivity (true positive rate, i.e., TP/P), specificity (true negative rate, i.e., TN/N), and accuracy, i.e., (TP+TN)/(P+N). For the current evaluation, P(positive) referred to extra-vesical bladder cancer, whereas N (negative) referred to intra-vesical bladder cancer. Also, the model was designed to maximize sensitivity, as it is clinically more important to identify cases if they have already turned from an intra-vesical (early stage) to an extra-vesical (late stage) bladder tumor. In other words, a false-positive (intra-vesical bladder tumor wrongly classified as an extra-vesical bladder tumor) is relatively more manageable than a false-negative (extra-vesical tumor wrongly identified as an intra-vesical tumor).

The radiogenomic staging model (RGs) was trained on all 28 cases of Data_{K} using the method explained above. The external validation was performed using all 12 test cases of Data_{N}. During external validation, the model used the same set of genetic and radiomic predictors identified in the training phase. The five machine learning classifiers generated satisfactory results, whereas the NB generated the highest mean classification sensitivity, specificity, and accuracy, reaching up to 94%, 88%, and 92%, respectively. The three matrices calculated for the training and testing of five classifiers are given in Table 2. The existing clinical staging method results in a sensitivity of 53.3%, a specificity of 87.5%, and an accuracy of 71.0%. The results indicate that RGs show more accurate staging performance than the existing clinical staging method.

TABLE 2
The results of the binary classification of bladder cancer
cases into low- and high-stage cancer using a radiogenomic model.
Five classifiers were tested on external data_{DataN}.

| | Validation (Data_{N}) | | |
|---|---|---|---|
| Classifier | Sen. | Spe. | Acc. |
| NB | 0.94 | 0.88 | 0.92 |
| SVM | 0.88 | 0.78 | 0.83 |
| DT | 0.89 | 0.72 | 0.80 |
| KNN | 0.78 | 0.72 | 0.75 |
| LR | 0.72 | 0.78 | 0.75 |

Two separate bladder staging models, one using genomic features (Gs) and the other using radiomic features (Rs), were trained and tested. The same five classifiers (Naïve Bayes, NB; Support Vector Machine, SVM; K-Nearest Neighbor, KNN; Logistic Regression, LR; and Decision Tree, DT) were trained for each of the genomic (GS) and radiomic (RS) models in conjunction with the RFE method. The significant features identified in the initial genomic and radiomic analysis using Data_{K} were used for the training of Gs and Rs, with a limit of up to 8 maximum features for each model. The GS model was tested using the genomic features in the three external models, that is, Data_{N}, Data_{W}, and Data_{B}, where the SVM obtained the highest mean classification sensitivity, specificity, and accuracy at 77%, 55%, and 67%, respectively. The RS model was tested using the MR images in Data_{N}, where the NB obtained the highest mean classification sensitivity, specificity, and accuracy at 77%, 72%, and 75%, respectively. The results show that the RGs model takes the lead by 25% and 17% from the Gs and Rs models, respectively, on mean classification accuracy, which supports the primary hypothesis of the study that integrating genomic and radiomic features can improve the staging accuracy of bladder cancer more than genomic or radiomic analysis alone.

All scripts for the described analysis were designed at the host institute and implemented on Matlab version 2023a. During external validation, all features extracted from previously unseen MR images lay within the expected range of values, and no infinity or undefined values were encountered. The model remained stable, and no thread crashed at any step throughout the analysis.

Currently, the clinical staging system for bladder cancer is based on the results of the transurethral resection of the bladder tumor/exam under anesthesia and imaging tests, specifically axial imaging of the abdomen and pelvis with CT-based imaging exams in the United States. As per the National Comprehensive Cancer Network (NCCN) guidelines, CT urography is recommended for patients suspected of bladder cancer, providing a detailed evaluation of the bladder, lymph nodes, potential metastases, and upper tract disease before TURBT. Two common techniques for CT urography are the single-bolus and split-bolus techniques, each with its advantages and considerations. While cystoscopy remains the gold standard for bladder evaluation, CT is widely used for the detection and staging of bladder urothelial carcinoma. CT urography, involving unenhanced, urothelial, and excretory phases, is valuable for both upper tract and bladder assessment, aiding in staging and post-treatment follow-up. The sensitivity and specificity of CT urography for detecting bladder UC are reported to be as high as 93% and 99%, making it particularly useful for identifying invasive tumors. Interestingly, in Japan, bladder cancer patients undergo both a CT scan to assess for nodal involvement/distant metastasis and an MRI scan to assess the bladder for specific T-stage or intra- or extra-vesical bladder cancer.

It is believed that MRI possesses better resolution than a CT scan and thus could be more accurate in staging bladder cancer. Furthermore, the inclusion of genomic features into MRI imaging holds the potential to greatly enhance bladder cancer staging, which could lead to improved medical decision-making. This is the first study showing the possibility that the automated integration of genetic expressions and MR features can efficiently assist in identifying the accurate stage of bladder cancer. While the naïve approach to distinguishing tumors of a low stage from a high stage primarily relies on tumor size, in this study, the textural and shape properties of the low- and high-stage bladder tumors (that apparently may have identical sizes) were thoroughly analyzed through radiomics to identify dissimilar features that are usually overlooked or uninterpretable by the naked eye. Several robust machine learning classifiers were deployed to explore the optimal combination of genetic and radiomic predictors to obtain the highest staging accuracy. Although the NB outperformed, the performances of other classifiers on three matrices were still comparable, showing the consistency of the identified significant features in genomic and radiomic analysis. A part of the reason for the enhanced performance of NB could be its probabilistic framework, effectively managing uncertainty in predictors from multiple omics, especially when dealing with limited training data. Moreover, the classification accuracy of the proposed radiogenomic model was on average 25% and 17% higher than that of exclusive genomic- and radiomic-based models, respectively.

To conduct these analyses, multiple retrospective datasets of gene expressions and MR scans of bladder cancer patients were collected. First, an extensive investigation of gene expressions and radiomic features of MR scans of bladder cancer patients was performed. The analysis identified several potential predictors specific to MIBC in both genetic and MR data, which validated our primary hypothesis. The analysis was followed by the development of a radiogenomic model for MIBC staging in which several machine learning algorithms were trained to perform automated binary classification of bladder cancer cases into either a low- or high-stage group using different combinations of the newly identified genetic and MR features. These algorithms were then evaluated at different performance bars to identify the one with the highest classification accuracy on the training data. The selected model was then validated against an independent external dataset, and the performance was estimated using different matrices. Moreover, two additional models, one based on genetic features and the other based on MR features alone, were trained and tested for MIBC staging. The performance of the proposed radiogenomic model was found to be significantly higher than the other two models, which strongly supports our proof of concept. This first study has high clinical application and encourages further investigation by replicating the model and validating its performance on large datasets.

The study's goal is to assist clinicians in better exploring and deciding optimal treatment, given a certain stage of cancer, to improve the outcome. The clinical stages associated with tumor growth, however, may be different, as stages Ta, Tis, and T1 are known as non-muscle-invasive bladder cancer (NMIBC), while stages T2-T4 are known as MIBC. However, the current study categorizes these stages into two major classes, that is, intra-vesical (Ta, Tis, T1, and T2) and extra-vesical (T3, and T4), based on the complexity of the subsequent treatment. For instance, patients at stage Ta or T1 (low grade) are primarily treated with TURBT, followed by immediate intra-vesical chemotherapy. These patients are prone to developing more low-stage tumors throughout their lives. Similarly, stage Ta, T1 (high grade), or Tis is commonly treated with a combination of TURBT and intra-vesical Bacillus Calmette-Guerin (BCG) immunotherapy or chemotherapy. It has been observed that such tumors may return at a more advanced stage. Patients at T2 or higher are given more aggressive and urgent treatment, including highly invasive surgeries. As we discussed, past studies have shown that the outcomes of bladder preservation with chemoradiotherapy are survival outcomes comparable to those of radical cystectomy when the patients suitable for the bladder-preserving strategy were properly selected, that is, not patients with T3 or T4 bladder cancer. Our study demonstrates improved clinical staging of bladder cancer patients when a new automated radiogenomic model is utilized. Such an automated radiogenomic model can be easily deployed in a clinical setting. Patients with a suspected bladder tumor seen on cystoscopy could undergo axial imaging of the pelvis with an MR scan (with contrast and T2 weighted images), followed by transurethral resection of the bladder tumor and RNA-seq or RT-PCR of a prescribed RNA signature. Subsequently, the automated radiogenomic model would more accurately stage bladder cancer patients, leading to improved outcomes.

Our study has three key contributions. First, a new set of genetic expressions potentially predictive of low and high stages of bladder cancer was identified that includes Luminal, Luminal differentiation, Neuroendocrine differentiation, Normal Basal Intermediate, and Normal CDH12. Second, it is the first time that the microlevel irregularities in the bladder tumors were investigated using radiomics of MR images, with the goal of uncovering patterns of tumor texture associated with the stage of bladder cancer. Various MR features potentially predictive of bladder cancer stages were identified, indicating that imaging can play an essential role in characterizing stages of MIBC. Third, the first automated radiogenomic model for bladder cancer staging was developed and validated on an independent dataset. The model outperformed the two models, each developed based on genomic and radiomic features alone. The RFE method, in conjunction with several common machine learning classifiers, efficiently identified the most optimal combination of radiomic and genomic features for accurate staging.

One of the study's strengths lies in the utilization of AI models, which systematically integrate complex genomic and radiomic data to characterize key features influencing disease progression, enabling accurate staging of bladder cancer. The increasing interest in employing AI for radiogenomics in various cancer-related objectives has been noted, owing to its superior performance that surpasses nearly all manual approaches. AI has proven instrumental in correlating radiomic patterns with specific genomic signatures across distinct cancer stages. Models employed in radiogenomic analysis for various cancers are described by Kang et al. in Front. Oncol. 8:228, 2018. While these models refine existing staging criteria and offer a more individualized assessment of a patient's disease status, challenges such as the need for larger datasets for model training and validation, the interpretability of AI-generated features, and the careful integration of these findings into clinical decision-making processes need consideration.

It is important to note that the model training prioritized maintaining higher sensitivity compared to specificity. Sensitivity, in this context, refers to accurately classifying cases into the high-stage category. This decision was intentional to minimize ‘false-negative’ instances (incorrectly identifying a ‘extra-vesical’ tumor as a ‘intra-vesical’ tumor). Justifying this choice ensures that cases classified as ‘intra-vesical’ but actually ‘extra-vesical’ (false-positive) undergo further evaluation, which may not have as severe consequences as the scenario where a ‘extra-vesical’ case is incorrectly classified as ‘intra-vesical’ (false-negative), potentially preventing the timely intervention to stop cancer growth to the advanced stage, that is, ‘extra-vesical’.

### Materials and Techniques

The study was performed after approval by the Cedars-Sinai Medical Center Institutional Review Board (IRB) (STUDY00001310), Kyoto University IRB (#G1301), and Nara Medical University IRB (#2967) under a request for waiver of consent on archived pathologic specimens and magnetic resonance imaging (MRI). Potential study subjects were identified by searching for both MRI and pathology data repositories from both Japanese centers for the years between 2010 and 2020. Each cases in both datasets came with a deidentified imaging report specifying the clinical stage of the cancer and the final pathologic stage obtained at the time of radical cystectomy. Next, we gathered a comprehensive collection of RNA and MRI data and bladder tumor formalin-fixed paraffin-embedded (FFPE) at the time of TURBT from these two centers, where MRI imaging of the bladder is routinely performed for staging bladder cancer, with one center serving as a training cohort and the other as a testing cohort. The training cohort consisted of 28 cases from the Kyoto University Hospital (termed Data_{K}), including both MRI scans and RNA sequencing (RNA-seq). The cases belong to 25 males and 3 females, with a median age of 78. Among these cases, 18 were classified as intra-vesicle tumor cases (Ta, Tis, T1, and T2), while the remaining 10 cases were categorized as extra-vesicle tumor cases (T3, T4, and N+ or M+). The testing cohort consisted of 12 cases from Nara Medical University Hospital (termed Data_{N}), including both MRI scans and RNA sequencing (RNA-seq). The cases belong to 11 males and 1 female, with a median age of 71. Out of these cases, 6 were classified as intra-vesicle tumor cases, while the remaining 6 cases were identified as extra-vesicle tumor cases. The subjects in both datasets belong to Asian ethnic groups.

Moreover, two public datasets, one published by Wrana J L. et al. in Eur Urol in 2014 (termed Data_{W}) (Liu et al., Eur Urol 2014, 66, 982-986) and the other by Jolanta Bondaruk et al. in iScience in 2022 (termed Data_{B}) (Bondaruk et al., iScience, 2022, 25, 104551), were also included in the study. The Data_{W} consists of the next-generation RNA-seq of archival formalin-fixed paraffin-embedded urothelial bladder cancer. The dataset consisted of 27 cases categorized as intra-vesicle tumor cases and 22 cases categorized as extra-vesicle tumor cases, whereas the Data_{B} consists of geographically annotated mucosal samples from human cystectomies performed in 9 patients with bladder cancer (3 basal and 6 luminal molecular subtypes) and 2 subjects with normal bladders. The samples ranged from normal urothelium (NU) to urothelial carcinoma (UC), obtained from individual patients, exhibiting varying degrees of dysplasia, including mild dysplasia, moderate dysplasia, severe dysplasia, and carcinoma in situ. These samples comprised microscopically normal urothelium, in situ preneoplastic conditions known as low-stage intra-urothelial neoplasia and high-grade intra-urothelial neoplasia, as well as UC. By incorporating these diverse datasets, the aim of the study was to provide a comprehensive and multi-dimensional understanding of bladder cancer at different clinical stages, leveraging RNA sequencing and MRI data to gain insights into the disease's underlying mechanisms and potential biomarkers.

As a part of preprocessing, the RNA isolation was performed on FFPE curls using the FormaPure XL RNA isolation kit (Beckman Coulter, Brea, CA, USA). Purified total RNA was tested for purity using the NanoDrop 8000 (ThermoFisher Scientific, Waltham, MA, USA), quantified using the Qubit Flex fluorometer (ThermoFisher Scientific), and the 2100 Bioanalyzer (Agilent Technologies, Santa Clara, CA, USA). Also, the library construction was performed using the Lexogen QuantSeq 3t mRNA-Seq Library Prep Kit FWD for Illumina. (Lexogen, Vienna, Austria). Briefly, all RNA samples were assessed for concentration using a Qubit fluorometer and for quality using the 2100 Bioanalyzer. Up to 100 ng of total RNA per sample was used for library preparation. Library concentration was measured with a Qubit fluorometer, and library size was measured on an Agilent 4200 TapeStation (Agilent Technologies). Libraries were multiplexed and sequenced on a NovaSeq 6000 (Illumina, San Diego, CA, USA) using 75 bp single-end sequencing. On average, about 10 million reads were generated from each sample.

### Radiogenomic Staging of Bladder Cancer

The following sections describe an automated methodology for radiogenomic-based staging of bladder cancers.

### Genomic Analysis of MIBC Staging

The purpose of the genomic analysis of patients with bladder cancer was to identify gene expressions that are significantly different between intra- and extra-vesicle tumors. For this, RNA-seq technology was employed to capture a comprehensive snapshot of the transcriptome. To ensure data reliability and accuracy, stringent quality control measures were implemented throughout the RNA-seq workflow. The data were ensured to be the most accurate by carefully evaluating the quality of the sequencing reads, assessing library complexity, and monitoring various metrics.

A differential analysis was conducted using the DESeq2 package in R to compare gene expression between low- and high-stage samples in the Data_{K}. DESeq2 employs empirical Bayes methods to accurately identify differentially expressed genes (DEGs) from count data. By analyzing DEGs, we gain insights into the molecular changes associated with disease progression and potential therapeutic targets or biomarkers. By overlapping the differentially expressed genes, we were able to categorize them separately based on their higher expression in intra-vs. extra-vesical cases.

To assess the relevance of these gene signatures, the ssGSEA algorithm was applied. The ssGSEA algorithm is a widely used computational method that quantifies the activity of predefined gene sets in individual samples. It measures the enrichment score of each gene set in each sample by comparing the ranks of the genes in the set to the ranks of all other genes in the sample. This approach enables us to evaluate the activity levels of the BLCA (Bladder Urothelial Carcinoma) stage-related gene signatures in each sample, providing insights into the underlying biology and potential clinical implications. By applying the ssGSEA algorithm to Data_{K}, we assessed the enrichment of the bladder cancer-stage-related gene signatures in different stages of bladder cancer. This analysis helped identify the gene sets that are significantly associated with specific stages of bladder cancer. Our focus was on a set of 75 bladder cancer-stage-related signatures (identified in multiple published references), with the goal of identifying and validating the most significant signatures while considering potential confounding effects stemming from patient-to-patient variability.

### Radiomic Analysis of Bladder Cancer Staging

Radiomic analysis is an approach to extracting and thoroughly analyzing the high-throughput image features of anatomical structures, including tumors, to assist in prediction, diagnosis, and prognosis. Using predefined mathematical quantities, radiomics can characterize tumor phenotypes based on complex multidimensional arrays of image-derived measurements.

An extensive radiomic analysis of bladder tumors using MR scans was performed to seek features that are significantly different between intra-vs. extra-vesical tumors. The analysis was performed using all 28 high-resolution T2-weighted MR scans in Data_{K}. Preprocessing included outlining the tumors in all MR images by two independent and experienced radiologists. Any labeling disagreements were resolved through consensus with a third radiologist. The ITK-snap version 3.0 software was used throughout the interactive labeling process. Each of the 28 images in Data_{K} was normalized (i.e., voxel values in each image were scaled between 0 and 1) before analysis using the min-max scaling. Several thousand radiomic features were then extracted from the outlined bladder tumors in each of these images. Each radiomic feature represented a unique MR image characteristic of the tumor and was expressed as a single numerical value calculated using a standard pre-defined mathematical formula. Various radiomic features were considered, including 15 First Order Statistics (e.g., Kurtosis, Percentiles, Range, etc.), 20 Gray-level Co-occurrence Matrix statistics (e.g., Cluster shade, Contrast, Autocorrelation, etc.), 15 Gray Level Run Length Matrix statistics (e.g., Run percentage, Run entropy, etc.), 14 Gray Level Size Zone Matrix (e.g., Zone percentage, Zone variance, etc.), 12 Gray Level Dependence Matrix (e.g., Small dependence emphasis, etc.), 20 Shape-based Features 2D/3D (e.g., Volume, Surface area, Sphericity, etc.), and 5 others (e.g., Complexity, Busyness, etc.). For example, to extract the signal intensity of the bladder tumor region, the mean gray level values of all voxels in the outlined boundary of the tumor in all slices of a three-dimensional bladder MR scan were considered.

All radiomic features were extracted using different combinations of three significantly important radiomic parameters, including bin size, kernel size, and angle. The Bin size determines the number of bins in the discretization process of the scan. The discretization decreases the chance of noise amplitude by transferring the continuous values of voxels into discrete counterparts, avoiding contrast variation among all MR scans acquired from different scanners. The Kernel size specifies the proximity around a voxel as a fixed window within which the spatial relationships of voxels with each other are calculated. The Angle determines the directions in which the spatial neighborhood is to be considered within the Kernel window. The choice of the three parameters has a great influence on the overall analysis. The Bin size used during feature extraction was between 2x=1 and 2x=8. The window size for the Kernel could take values between 1 and 5, whereas the Angle in all four quadrants was considered. Thus an exhaustive analysis was performed using all possible combinations of the three parameters, which yielded nearly 6000 radiomic features from each of the 28 images in Data_{K}. All features were extracted using an in-house radiomic feature-extraction application.

### Radiogenomic Modeling for Bladder Cancer Staging

A machine learning model was developed that performs automated staging of bladder cancer through the integration of the above genomic and radiomic predictors. A systematic approach was adopted to ensure that the proposed model selects the minimal set of reliable and consistent predictors from both data categories to avoid overfitting and increasing generalizability without compromising the overall performance of the model.

The preprocessing steps include identifying a subset of the significant features identified in the radiomic analysis that show the highest predictive strength. Such features were obtained in two phases: the first phase consisted of randomly selecting one of the multiple features that differed only in the combination of the radiomic parameters these were obtained with. For example, if multiple instances of a feature (e.g., contrast) were identified as significant (each with a unique combination of Bin size, Kernel size, and Angle), then only one of the instances would be selected and the rest would be ignored. In the second phase, the subset of features was further narrowed to a sub-subset by choosing the features that show significance at a minimal p-value such that the number of features in the final set corresponds to the number of features identified as significant in the genomic analysis. The final set of radiomic features consisted of 12 unique features identified at p=0.01, as Energy, Inverse gaussian left, Inverse gaussian left focus, Gaussian right polar, Cluster promin, Cluster shade, Cluster trend, Homogeneity, Autocorrelation, Gaussian, Gaussian right focus, and Gaussian right polar.

Subsequently, several common fully supervised machine learning classifiers were trained to perform automated staging of bladder cancer using both sets of significant features. The staging was expressed as a binary classification problem, with intra-vs. extra-vesical as two possible categories. The NB classifier worked based on the Bayes theorem with the “naive” assumption of conditional independence between every pair of features (e.g., feature independence within and across radiomic and genetic feature sets). The KNN performed classification using the assumption of the proximity of values for features from a class (e.g., values of radiomic features from low-stage tumors were expected to be closer than those from high-stage tumors). The SVM performed classification by specifying among hyperplanes and separating the radiomic and genomic features of two classes such that the margins of the planes are maximized. The LR worked based on the linear regression technique, which finds an optimal boundary between two sets of features using a logistic sigmoid function to avoid outliers. The DT classifier worked by specifying nodes (each denoting a radiomic/genomic feature), branches (each denoting a decision of the test), and terminal nodes (each representing one of the two stages of bladder cancer).

These algorithms were chosen for analysis due to their widespread acceptance in binary classification problems and frequent utilization for their performance. These algorithms have been employed in numerous studies to identify the critical aspects of patients' conditions and model the progression of diseases following treatment using intricate health information and medical datasets. These algorithms have demonstrated stability in addressing cancer staging problems, including recurrence of stage IV colorectal cancer, cancer genomics and subtyping of breast cancer, and detection of early stages of pancreatic cancer. Moreover, given the pilot nature of this study and the small dataset, we steered clear of advanced or complex algorithms to prevent potential overfitting.

Finally, the Recursive Feature Elimination (RFE) method was deployed in conjunction with the five mentioned classifiers to eliminate the relatively weak features by comparing the overall training accuracy achieved by each classifier using different combinations of features. A classifier could select up to a maximum of 8 significant features from each of two categories (i.e., genomics and radiomics) while maximizing the classification accuracy. Any features from each of the two categories were counted with equal weight.

### Statistical Analysis

Statistical analyses were performed with R version 4.0.3 (www.r-project.org/, accessed on 1 Aug. 2021), and the visualization of heat maps was achieved using the Complex Heat Maps Bioconductor package. To assess differences among multiple groups for continuous variables, Kruskal-Wallis tests were applied. Additionally, the Gene Set Enrichment Analysis of tumor single-cell subtype signatures was conducted using the single sample Gene Set Enrichment Analysis (ssGSEA) function from the R package GSVA. All p-values were two-sided, and p<0.05 was considered to indicate a statistically significant difference.

Overall, this study provides a proof-of-concept with promising results of a radiogenomic approach for bladder cancer staging and encourages researchers to further validate the proposed model on large datasets. The methodology ensured avoiding overfitting by restricting the number of predictors used in the model. Furthermore, with the certainty that at least 40% of the enrolled subjects are from each of the low- and high-stage groups, a relatively small number of cases was sufficient to develop the classification model. The lack of specific and well-established tumor characteristics to recognize its stage leads to inefficient management of bladder cancer. To our knowledge, the proposed data structure has not been used previously for bladder cancer staging. Taken together, we provide herein a robust method in prospective studies that helps increase the rate of efficient bladder cancer staging.

Various embodiments of the invention are described above in the Detailed Description. While these descriptions directly describe the above embodiments, it is understood that those skilled in the art may conceive modifications and/or variations to the specific embodiments shown and described herein. Any such modifications or variations that fall within the purview of this description are intended to be included therein as well. Unless specifically noted, it is the intention of the inventors that the words and phrases in the specification and claims be given the ordinary and accustomed meanings to those of ordinary skill in the applicable art(s).

The foregoing description of various embodiments of the invention known to the applicant at this time of filing the application has been presented and is intended for the purposes of illustration and description. The present description is not intended to be exhaustive nor limit the invention to the precise form disclosed and many modifications and variations are possible in the light of the above teachings. The embodiments described serve to explain the principles of the invention and its practical application and to enable others skilled in the art to utilize the invention in various embodiments and with various modifications as are suited to the particular use contemplated. Therefore, it is intended that the invention not be limited to the particular embodiments disclosed for carrying out the invention.

While particular embodiments of the present invention have been shown and described, it will be obvious to those skilled in the art that, based upon the teachings herein, changes and modifications may be made without departing from this invention and its broader aspects and, therefore, the appended claims are to encompass within their scope all such changes and modifications as are within the true spirit and scope of this invention. It will be understood by those within the art that, in general, terms used herein are generally intended as “open” terms (e.g., the term “including” should be interpreted as “including but not limited to,” the term “having” should be interpreted as “having at least,” the term “includes” should be interpreted as “includes but is not limited to,” etc.). As used herein the term “comprising” or “comprises” is used in reference to compositions, methods, and respective component(s) thereof, that are useful to an embodiment, yet open to the inclusion of unspecified elements, whether useful or not. It will be understood by those within the art that, in general, terms used herein are generally intended as “open” terms (e.g., the term “including” should be interpreted as “including but not limited to,” the term “having” should be interpreted as “having at least,” the term “includes” should be interpreted as “includes but is not limited to,” etc.). Although the open-ended term “comprising,” as a synonym of terms such as including, containing, or having, is used herein to describe and claim the invention, the present invention, or embodiments thereof, may alternatively be described using alternative terms such as “consisting of” or “consisting essentially of.”

What is claimed is:

1. A method of determining severity or stage of bladder cancer in a subject in need thereof and treating the subject in need thereof, comprising:
i) measuring in a biological sample of the subject an expression level of one or more marker genes of HOXB5, DHRS3, FABP4, TAGLN2, HIST1H1D, HIST1H2BD, H2AFX, CLDN3, and PLAUR; and/or
ii) performing radiomic analysis on a magnetic resonance (MR) image associated with the bladder of the subject by extracting from a region of interest (ROI) in the MR image one or more radiomic features of Energy, Cluster Prominence, Cluster Shade, Cluster Tendency, Homogeneity, Autocorrelation, Inverse Gaussian Left, Inverse Gaussian Left Focus, Gaussian Right Polar, Gaussian, Gaussian Right Focus, and Gaussian Right Polar;
and
providing a treatment comprising cystectomy to the subject if the subject is indicated as having a late stage bladder cancer, or providing a treatment comprising one or more of chemotherapy, radiotherapy, immunotherapy, and trans urethral resection of bladder tumour and excluding cystectomy if the subject is indicated as having an early stage bladder cancer;
wherein the subject is indicated as having a late stage bladder cancer when the expression level of the one or more of the TAGLN2, the HIST1H1D, the HIST1H2BD, the H2AFX, the CLDN3, and the PLAUR, if measured, is higher, the expression level of the one or more of the HOXB5, the DHRS3, and the FABP4, if measured, is lower, and the extracted one or more radiomic features are higher, compared to respective ones in a reference subject having only a T0, T1, or T2 stage bladder cancer,
wherein the subject is indicated as having an early stage bladder cancer when the expression level of the one or more of the HOXB5, the DHRS3, and the FABP4, if measured, is higher, the expression level of the one or more of the TAGLN2, the HIST1H1D, the HIST1H2BD, the H2AFX, the CLDN3, and the PLAUR, if measured, is lower, and the extracted one or more radiomic features are lower, compared to respective ones in a reference subject having a T3 or T4 stage bladder cancer.

2. The method of claim 1, further comprising measuring in the biological sample expression levels of all or one or more genes from one or more of a plurality of gene sets selected from the group consisting of Basal, Basal Differentiation, Base Excision Repair, Cell Cycle, Homologous Recombination, MicroRNAs in Cancer, Oocyte Meiosis, p53 Signaling Pathway, Progesterone-mediated Oocyte Maturation, Pyrimidine Metabolism, Luminal, Luminal differentiation, Neuroendocrine differentiation, Normal Basal Intermediate, and Normal CDH12,
wherein the gene set Basal comprises genes of CD44, CDH3, KRT1, KRT14, KRT16, KRT5, KRT6A, KRT6B, KRT6C, SFN, S100A8, PI3, S100A7, CSTA, SERPINB2, CKS2, CCNB1, CC2, KIF23, and PMAIP1,
the gene set Basal Differentiation comprises genes of LYNX1, SCEL, CTB-113P19.4, S100A8, SLP1, S100A9, PI3, CEACAM6, VSNL1, SERPINB2, CSTA, FGFBP1, SPRR1B, DSG3, KRT1, S100A7, CLCA2, LGALS7, SPRR2B, KRT6A, KRT6B, SFN, SERPINB3, KRT16, SERPINB3, SERPINB13, SERPINB4, KRT4, SPRR1A, KRT6A, ABCA12, SPRR3, and TME,
the gene set Base Excision Repair comprises genes of FEN1, LIG1, NEIL3, PARP1, PARP2, PCNA, POLE, POLE2, and UNG,
the gene set Cell Cycle comprises genes of BUB1, BUBIB, CC2, CCNB2, CCNE1, CCNE2, CDC20, CDC25A, CDC25C, CDC6, CDK1, CDK2, CDKN2A, DBF4, E2F1, E2F2, ESPL1, MAD2L1, MAD2L2, MCM2, MCM4, MCM6, MCM7, ORC1, ORC6, PC, PLK1, SKP2, SMC3, TFDP1, TTK, YWHAB, ABL1, APC1, APC10, APC11, APC13, APC2, APC4, APC5, APC7, ATM, ATR, BUB1, BUBIB, BUB3, CC1, CC2, CCNB1, CCNB2, CCNB3, CCND1, CCND2, CCND3, CCNE1, CCNE2, CCNH, CDC14A, CDC14B, CDC16, CDC20, CDC23, CDC25A, CDC25B, CDC25C, CDC26, CDC27, and CD,
the gene set Homologous Recombination comprises genes of BLM, BRCA2, EME1, RAD51, RAD51C, RAD54L, RPA1, RPA3, TOP3A, XRCC2, and XRCC3,
the gene set MicroRNAs in Cancer comprises genes of BRCA1, CCNE1, CCNE2, CDC25A, CDC25C, CDCA5, CDKN2A, DNMT1, E2F1, E2F2, EZH2, KIF23, STMN1, and TRIM71,
the gene set Oocyte Meiosis comprises gene of AURKA, BUB1, CCNB2, CCNE1, CCNE2, CDC20, CDC25C, CDK1, CDK2, ESPL1, FBXO5, MAD2L1, MAD2L2, PLK1, SGOL1, SMC3, and YWHAB,
the gene set p53 Signaling Pathway comprises genes of CCNB2, CCNE1, CCNE2, CDK1, CDK2, CDKN2A, GTSE1, PPMID, RFWD2, and RRM2,
the gene set Progesterone.mediated Oocyte Maturation comprises genes of BUB1, CCNA2, CCNB2, CDC25A, CDC25C, CDK1, CDK2, MAD2L1, MAD2L2, and PLK1, and
the gene set Pyrimidine Metabolism comprises genes of CTPS1, DTYMK, POLA2, POLE, POLE2, PRIM1, PRIM2, RRM2, and TYMS.

3. The method of claim 2, wherein the expression levels of the all or one or more genes from each of the Basal, the Basal Differentiation, the Base Excision Repair, the Cell Cycle, the Homologous Recombination, the MicroRNAs in Cancer, the Oocyte Meiosis, the p53 Signaling Pathway, the Progesterone-mediated Oocyte Maturation, and the Pyrimidine Metabolism, gene sets if measured are higher in the subject indicated as having a late stage bladder cancer, compared to respective ones in the reference subject having only a T0, T1, or T2 stage bladder cancer; and
wherein the expression levels of the all or one or more genes from each of the Luminal, the Luminal differentiation, the Neuroendocrine differentiation, the Normal Basal Intermediate, and the Normal CDH12 are higher in the subject indicated as having an early stage bladder cancer, compared to respective ones in the reference subject having a T3 or T4 stage bladder cancer.

4. The method of claim 1, wherein the Energy is computed as energy=Σᵢ₌₁^{N}^{ₚ}(X(i)+c)²,
the Autocorrelation is computed as autocorrelation=Σᵢ₌₁^{N}^{_{g}}Σⱼ₌₁^{N}^{_{g}}p(i,j)ij,
the Cluster Prominence is computed as cluster prominence=Σᵢ₌₁^{N}^{_{g}}Σⱼ₌₁^{N}^{_{g}}(i+j−μₓ−μ_{y})⁴p(i,j),
the Cluster Shade is computed as cluster shade=Σᵢ₌₁^{N}^{_{g}}Σⱼ₌₁^{N}^{_{g}}(i+j−μₓ−μ_{y})³p(i,j),
the Cluster Tendency is computed as cluster tendency=Σᵢ₌₁^{N}^{_{g}}Σⱼ₌₁^{N}^{_{g}}(i+j−μₓ−μ_{y})²p(i,j),
the Homogeneity is computed as
$$homogeneity = {\sum }_{i = 1}^{{N}_{g}} {\sum }_{j = 1}^{{N}_{g}} \frac{p ( i , j )}{1 + \vert i - j \vert} ,$$
the Inverse Gaussian Left is computed as Inverse Gaussian Left=Σᵢ₌₁ⁿΣⱼ₌₁ⁿtempᵢⱼ·glcmᵢⱼ,
the Inverse Gaussian Left Focus is computed as Inverse Gaussian Left Focus=Σᵢ₌₁ⁿΣⱼ₌₁ⁿtempᵢⱼ·glcmᵢⱼ,
the Inverse Gaussian Right Polar is computed as Inverse Gaussian Right Polar=Σᵢ₌₁ⁿΣⱼ₌₁ⁿtempᵢⱼ·glcmᵢⱼ,
the Gaussian Right Polar is computed as Gaussian Right Polar=Σᵢ₌₁ⁿΣⱼ₌₁ⁿtempᵢⱼ·glcmᵢⱼ,
the Gaussian Right Focus is computed as Gaussian Right Focus=Σᵢ₌₁ⁿΣⱼ₌₁ⁿtempᵢⱼ·glcmᵢⱼ, and
the Gaussian is computed as Gaussian=Σᵢ₌₁ⁿΣⱼ₌₁ⁿtempᵢⱼ·glcmᵢⱼ,
wherein the ROI contains a set of Nₚ voxels from 1 to Nₚ; X(i) is a gray level intensity of voxel i, c is a pre-determined constant such that (X(i)+c) is not a negative value; N_{g} is number of non-zero bins; p(i, j) is a normalized co-occurrence matrix and equal to P(i,j)/ΣP(i,j), P(i,j) is the co-occurrence matrix for an arbitrary δ and θ, and (i, j)^{th} element is a number of times the combination of levels i and j occur in two pixels separated by a distance of δ pixels along angle θ; μₓ is mean gray level intensity of pₓ and defined as μₓ=Σᵢ₌₁^{N}^{_{g}}(i)i; μ_{y} is mean gray level intensity of p_{y} and defined as μ_{y}=Σⱼ₌₁^{N}^{_{g}}(j)j.

5. The method of claim 1, wherein the subject in need thereof is a human with suspected bladder tumor or human with a sessile appearing bladder mass.

6. The method of claim 1, wherein the biological sample comprises a bladder biopsy, a resected bladder tumor specimen, or a mucosal sample from cystectomy.

7. The method of claim 1, wherein the measuring of a gene expression level comprises performing single-cell RNA sequencing.

8. The method of claim 1, wherein the method comprises the measuring of the expression level in the biological sample and the providing of the treatment.

9. The method of claim 1, wherein the method comprises the performing of the radiomic analysis and the providing of the treatment.

10. The method of claim 1, wherein the method comprises the measuring of the expression level and the performing of the radiomic analysis, followed by the providing of the treatment.

11. A method of treating a subject in need thereof, comprising:
administering chemotherapy, radiation, immunotherapy, and/or performing transurethral resection of bladder tumor (TURBT), but not cystectomy, in a subject indicated as having an early stage bladder cancer, or
performing cystectomy in a subject indicated as having a late stage bladder cancer,
wherein the subject is indicated as having an early stage bladder cancer when expression level of one or more of genes HOXB5, DHRS3, and FABP4 in a biological sample of the subject is higher, expression level of one or more of genes TAGLN2, HIST1H1D, HIST1H2BD, H2AFX, CLDN3, and PLAUR in the biological sample is lower, and one or more radiomic features in a magnetic resonance image associated with a bladder of the subject is lower, compared to respective ones in a reference subject having a T3 or T4 stage bladder cancer,
wherein the subject is indicated as having a late stage bladder cancer when expression level of one or more of the genes TAGLN2, HIST1H1D, HIST1H2BD, H2AFX, CLDN3, and PLAUR in the biological sample is higher, expression level of one or more of the genes HOXB5, DHRS3, and FABP4 in the biological sample is lower, and the one or more radiomic features in the magnetic resonance image associated with the bladder of the subject is higher, compared to respective ones in a reference subject having a T0, T1 or T2 stage bladder cancer, and
wherein the one or more radiomic features comprises Energy, Cluster Prominence, Cluster Shade, Cluster Tendency, Homogeneity, Autocorrelation, Inverse Gaussian Left, Inverse Gaussian Left Focus, Gaussian Right Polar, Gaussian, Gaussian Right Focus, and Gaussian Right Polar.

12. The method of claim 11, wherein the subject indicated as having an early stage bladder cancer further has:
a higher expression level of all or one or more genes from one or more of gene sets Luminal, Luminal differentiation, Neuroendocrine differentiation, Normal Basal Intermediate, and Normal CDH12, and/or a low expression level of all or one or more genes from one or more of gene sets Basal, Basal Differentiation, Base Excision Repair, Cell Cycle, Homologous Recombination, MicroRNAs in Cancer, Oocyte Meiosis, p53 Signaling Pathway, Progesterone-mediated Oocyte Maturation, and Pyrimidine Metabolism, in the biological sample compared to respective ones in the reference subject having a T3 or T4 stage bladder cancer.

13. The method of claim 11, wherein the subject indicated as having a late stage bladder cancer further has:
a higher expression level of all or one or more genes from one or more of gene sets Basal, Basal Differentiation, Base Excision Repair, Cell Cycle, Homologous Recombination, MicroRNAs in Cancer, Oocyte Meiosis, p53 Signaling Pathway, Progesterone-mediated Oocyte Maturation, and Pyrimidine Metabolism, and/or a low expression level of all or one or more genes from one or more of gene sets Luminal, Luminal differentiation, Neuroendocrine differentiation, Normal Basal Intermediate, and Normal CDH12, in the biological sample compared to respective ones in the reference subject having a T0, T1 or T2 stage bladder cancer.

14. The method of claim 11, wherein the subject in need thereof is a human, and the biological sample comprises a bladder biopsy or a resected bladder tumor specimen.

15. A method of monitoring response to a therapy and providing subsequent treatment in a subject with a bladder cancer, comprising:
providing the therapy to the subject with a bladder cancer, and,
in response to the therapy,
i) measuring in a biological sample of the subject an expression level of one or more marker genes of HOXB5, DHRS3, FABP4, TAGLN2, HIST1H1D, HIST1H2BD, H2AFX, CLDN3, and PLAUR;
ii) performing radiomic analysis on a magnetic resonance (MR) image associated with the bladder of the subject by extracting from a region of interest (ROI) in the MR image one or more radiomic features of Energy, Cluster Prominence, Cluster Shade, Cluster Tendency, Homogeneity, Autocorrelation, Inverse Gaussian Left, Inverse Gaussian Left Focus, Gaussian Right Polar, Gaussian, Gaussian Right Focus, and Gaussian Right Polar; and/or
iii) measuring in the biological sample expression levels of all or one or more genes from one or more of a plurality of gene sets selected from the group consisting of Basal, Basal Differentiation, Base Excision Repair, Cell Cycle, Homologous Recombination, MicroRNAs in Cancer, Oocyte Meiosis, p53 Signaling Pathway, Progesterone-mediated Oocyte Maturation, Pyrimidine Metabolism, Luminal, Luminal differentiation, Neuroendocrine differentiation, Normal Basal Intermediate, and Normal CDH12,
and
continuing the therapy, optionally in a lower dose than before, as a subsequent treatment for the subject if the expression level of the HOXB5, the DHRS3, and the FABP4 if measured has increased, the expression level of the TAGLN2, the HIST1H1D, the HIST1H2BD, the H2AFX, the CLDN3, and the PLAUR if measured has decreased, the one or more radiomic features if measured has lowered, the expression levels of the all or one or more genes from the gene sets Luminal, Luminal differentiation, Neuroendocrine differentiation, Normal Basal Intermediate, and Normal CDH12 if measured has increased, and the expression levels of the all or one or more genes from the gene sets Basal, Basal Differentiation, Base Excision Repair, Cell Cycle, Homologous Recombination, MicroRNAs in Cancer, Oocyte Meiosis, p53 Signaling Pathway, Progesterone-mediated Oocyte Maturation, and Pyrimidine Metabolism if measured has decreased, in response to the therapy, or
providing additional therapies, increasing doses, and/or replacing the therapy with radical cystectomy in the subject as subsequent treatment if the expression level of the TAGLN2, the HIST1H1D, the HIST1H2BD, the H2AFX, the CLDN3, and the PLAUR if measured has increased, the expression level of the HOXB5, the DHRS3, and the FABP4 if measured has decreased, the one or more radiomic features if measured has increased, the expression levels of the all or one or more genes from the gene sets Basal, Basal Differentiation, Base Excision Repair, Cell Cycle, Homologous Recombination, MicroRNAs in Cancer, Oocyte Meiosis, p53 Signaling Pathway, Progesterone-mediated Oocyte Maturation, and Pyrimidine Metabolism if measured has increased, and the expression levels of the all or one or more genes from the gene sets Luminal, Luminal differentiation, Neuroendocrine differentiation, Normal Basal Intermediate, and Normal CDH12 if measured has decreased, in response to the therapy.
